(19) United States
(12) Patent Application Publication
(10) Pub. No.: US 2012/0208266 A1
(43) Pub. Date: Aug. 16, 2012

(54) ENZYME CLEAVABLE CELL RELEASE POLYMERIC SURFACE

(76) Inventors: Dana Craig Bookbinder, Corning, NY (US); Arthur Winston Martin, Horseheads, NY (US); Jodelle Karen Nelson, Painted Post, NY (US); Shawn Michael O'Malley, Horseheads, NY (US); Meenal Pauddar Soni, Painted Post, NY (US)

(21) Appl. No.: 13/367,661

(22) Filed: Feb. 7, 2012

Related U.S. Application Data
(60) Provisional application No. 61/441,891, filed on Feb. 11, 2011.

(51) Int. Cl. C12M 3/00

(52) U.S. Cl. 435/304.1; 435/289.1; 435/305.2; 435/305.1

(57) ABSTRACT

Disclosed herein are cell culture surfaces comprising a substrate and a polymer layer on the substrate where the polymer layer contains enzyme-cleavable amino acid sequences and cell adhesive peptide sequences.

### CROSS-REFERENCE TO RELATED APPLICATIONS

This application claims the benefit of priority under 35 U.S.C. §119 of U.S. Provisional Application Ser. No. 61/441,891 filed on Feb. 11, 2011.

### SEQUENCE LISTING

The instant application contains a Sequence Listing which has been submitted in ASCII format via EFS-Web and is hereby incorporated by reference in its entirety. Said ASCII copy, created on Mar. 9, 2012, is named SP1117US.txt and is 19 kb in size.

### FIELD

The present disclosure relates to peptide-conjugated polymeric cell culture surfaces which can be activated by proteolytic enzymes to release cells. More particularly, the disclosure relates to polymeric surfaces which contain sequences that are amenable for enzymatic cleavage, conjugated to cell adhesion peptides.

### BACKGROUND

In the field of cell culture, researchers are seeking cell culture surfaces which improve cell characteristics such as cell growth in culture. Reducing potentially contaminating ingredients such as serum or cell extracts is also desirable. Synthetic polymeric surfaces have been used for cell culture. In some cases, these polymeric surfaces have been formed from monomers containing amino acids or peptides. Acrylated or methacrylated amino acids have been used to form cell culture surfaces.

For example, Hem and Hubbell (Diane L. Hem and Jeffrey A. Hubbell; Incorporation of Adhesion Peptides into Nonadhesive Hydrogels useful for Tissue Resurfacing; J. Biomed. Mater. Res. 39, 266 (1998)) disclosed the formation of a (meth)acrylated peptide, including an adhesion peptide having an RGD sequence, by functionalizing the amine terminus of the peptide with an acrylate moiety. These functionalized peptides were then copolymerized with polyethylene glycol (PEG) or PEG diacrylate to form hydrogel cell culture surfaces having incorporated cell adhesion sequences.

Successful culture of difficult-to-culture cells requires that cell culture surfaces be tailored to accommodate the particular requirements of these cells. Bone cells, for example, prefer to be cultured in the presence of hydroxyapatite surfaces such as those discussed in Song et al. Song et al (Jie Song, Vienghkam Malathong, Carolyn R. Bertozzi, Mineralization of Synthetic Polymer Scaffolds: A Bottom-Up Approach for the Development of Artificial Bone, J. Am. Chem. Socl., 2005, 127, 3366-3372) disclosed the use of anionic groups such as methacrylated GLY (GlyMA), SER (SerMA), ASP (SerMA) and GLU (GluMA), and a methacrylated amino acid sequence RGD, a known cell adhesive monomer, to form a polymeric hydro gel containing 2-hydroxyethyl methacrylate (HEMA) or 2-hydroxyethyl dimethacrylamide (HEMAm) and cross-linkers ethylene glycol dimethacrylate (EGDMA) or ethylene glycol dimethacrylamide (EGDMAm). These anionic functionalized hydrogels were then used to provide a substrate for hydroxyapatite mineralization, after exposure of the hydrogel to urea. The negatively charged monomers, along with hydroxyethyl ester side chains of pHEMA that may have been hydrolyzed during the mineralization process, provided CA^{2+ binding sites and allowed for the formation of a mineralized hydroxyapatite cell culture surface.}

Ciucurel and Sefton (Ema C. Ciucurel and Michael V. Sefton; A Poloxamine-Polylysine Acrylate Scoffold for Modular Tissue Engineering; J. Biomaterials Science, 2010 DOI:10.1163/092050610X541133) disclosed the use of acrylated polylysine polymerized with poloxamine to form a poloxamine-polylysine acrylate (PPA) photopolymerized polymer. PPA hydrogels were able to support the proliferation of human microvascular endothelial cells (HMEC-1, a cell line) in culture.

Melkoumian et. al. (Jennifer L. Weber, David M. Weber, Andrei G. Fadeev, Yue Zhou, Paula Dolley-Sonneville, Jiwei Yang, Liqun Qui, Catherine A. Priest, Christopher Shogbon, Arthur W. Martin, Jodelle Nelson, Peter West, James P. Beltzer, Santona Pal and Ralph Brandenberger), Synthetic peptide-Acrylate Surfaces for Long-Term Self-Renewal and Cardiomyocyte Differentiation of Human Embryonic Stem Cells, (2010) Nature Biotechnology, Vol. 28, Number 6, 606-610 disclosed peptide-acrylate surface for long-term culture and differentiation of cardiomyocytes derived from human embryonic stem cells.

### BRIEF DESCRIPTION OF THE DRAWINGS

FIG. 1 is a graphic illustrating an embodiment of the cell culture surface disclosed herein.

FIG. 2 is a graphic illustration of an embodiment of the cell culture surface disclosed herein.

FIG. 3 is a flow chart showing an embodiment of a method of making cell culture surfaces.

FIG. 4A-H are photographs showing morphology of human mesenchymal stem cells, cultured on a PARG-1-VN embodiment of the cell culture surface disclosed herein, after treatment with a proteolytic enzyme, at zero seconds (A), forty seconds (B), sixty seconds (C), ninety seconds (D), one hundred twenty seconds (E), 2 minutes and forty five seconds (F), three minutes and thirty seconds (G) and after removal of cells (H).

FIG. 5A-H are photographs showing morphology of human mesenchymal stem cells, cultured on a PARG-1-VN embodiment of the cell culture surface disclosed herein, after treatment with Trypsin and 1 mM EDTA, at zero seconds (A), forty seconds (B), sixty seconds (C), ninety seconds (D), one hundred twenty seconds (E), 2 minutes and forty five seconds (F), three minutes and thirty seconds (G) and after removal of cells (H).

FIG. 6 A-H are photographs showing morphology of human mesenchymal stem cells, cultured on a PLYS-1-VN embodiment of the cell culture surface disclosed herein, after treatment with a proteolytic enzyme, at zero seconds (A), forty seconds (B), sixty seconds (C), ninety seconds (D), one hundred twenty seconds (E), 2 minutes and thirty five seconds (F), three minutes and thirty seconds (G) and after removal of cells (H).

FIG. 7 A-H are photographs showing morphology of human mesenchymal stem cells, cultured on PLYS-1-VN embodiment of the cell culture surface disclosed herein, after treatment with Trypsin and 1 mM EDTA, at zero seconds (A), forty seconds (B), sixty seconds (C), ninety seconds (D), one hundred twenty seconds (E), 2 minutes and thirty five seconds (F), three minutes and thirty seconds (G) and after removal of cells (H).

FIG. 8A-D are photographs showing morphology of human mesenchymal stem cells, cultured on comparative example of a cell culture surface (Poly-D-Lysine, available from BD biosciences, Franklin Lakes, JN) after one day (A-C) and after four days (D) of culture. FIG. 4 illustrates that human mesenchymal stem cells did not adhere to the Poly-D-Lysine substrate.

FIG. 9 A-P are photographs showing morphology of human mesenchymal stem cells, cultured on comparative example of a cell culture surface (Synthemax™ available from Corning Incorporated, Corning, N.Y.) after treatment with a proteolytic enzyme, at time zero seconds (A); one minute (B); two minutes (C); three minutes (D); four minutes (E); five minutes (F); six minutes (G); after gentle tapping after six minutes (H); after eight minutes (I); after gentle tapping after eight minutes (K); after eleven minutes (L), after 12 minutes (M); after thirteen minutes (N); after gentle tapping after thirteen minutes (O); and after fifteen minutes (P).

### DETAILED DESCRIPTION

Disclosed herein are cell culture surfaces that are both amenable to cell culture of adherent cell types, and also amenable to release of adherent cell types. That is, in embodiments, the cell culture surface disclosed herein provides a surface to which cells will adhere. And, these cell culture surfaces also provide a mechanism for the release of cells, by enzyme cleavage of enzyme-cleavable peptide sequences present in the polymeric material of the cell culture surfaces.

Synthetic polymer films offer many advantages over animal derived coatings for cell culture including scalability, long term shelf-life, lot to lot variability control and lower costs of manufacture. However, one problem has been the ability to effectively release cells from some synthetic polymer surfaces. Often cells are released from surfaces by use of enzymes. Alternatively, surfaces have been prepared that rely on the removal of ions like calcium and magnesium, or temperature changes to alter the physical characteristics of the cell culture surface and allow cells to release from a surface.

Desirable cell release attributes of synthetic cell culture surfaces include reduced time to induce cell release, techniques that do not harm the external membrane and membrane protein structures of cells, and surfaces that allow efficient recovery of cells. These attributes are especially important in the area of stem cell therapy, since extended exposure of cells to enzymatic agents can affect cell viability, and yield of cells released from surfaces is especially important.

Disclosed herein are enzyme-activated synthetic cell culture surfaces which have a polymer layer on a substrate, the polymer layer comprising a proteolytic enzyme-cleavable amino acid sequence; and a cell adhesive peptide bound to the polymer layer.

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense. All scientific and technical terms used herein have meanings commonly used in the art unless otherwise specified. The definitions provided herein are to facilitate understanding of certain terms used frequently herein and are not meant to limit the scope of the present disclosure. As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise. As used herein, “have”, “having”, “include”, “including”, “comprise”, “comprising” or the like are used in their open ended sense, and generally mean “including, but not limited to.”

In embodiments, the polymer layer is formed from a functionalized proteolytic enzyme-cleavable peptide monomer that can be described by Formula 1:

Z-Xaaₙ-Z¹ₙ₁ Formula 1

wherein Z and Z¹ are polymerization moieties and n1 is an integer of 0 or 1; wherein Xaa is each independently an amino acid and n is an integer from 2 to 100 and wherein Xaaₙ is an amino acid sequence that is cleavable by a protease enzyme. In embodiments, the carboxyl terminus of the amino acid sequence is amidated. These polymerization moieties form the “functionalization” that allows for polymerization of the monomers. That is, a “functionalized” monomer has a polymerization moiety.

In embodiments, functionalized proteolytic enzyme-cleavable peptide monomer or monomers may be described by Formula 1 (Z-Xaaₙ-Z¹ₙ₁) where Z and Z¹ are polymerization moieties which are, for example, α, β unsaturated ethylenically unsaturated groups which include, for example, acrylate, methacrylate, acrylamide, methacrylamide, maleimide or fumarate groups. Epoxide, methacryoyl or vinyl functional groups may also be polymerizable moieties. One such polymerization moiety (Z) is bound to the carboxyl end of the peptide. In embodiments, a polymerization moiety may be bound to the epsilon amine of the lysine sidechain, as shown in Formulas 4 and 5, for example. In embodiments these polymerization moieties can form polymers upon exposure to an energy source. For example, in embodiments, the polymerization moiety may be polymerizable through exposure to light or temperature change.

In embodiments, each Xaa is independently an amino acid. The term “independently” is used herein to indicate that each Xaa may differ from other Xaa amino acids. In embodiments, at least one Lys amino acid is present, and at least one Lys amino acid of Xaaₙ comprises a polymerization moiety at the epsilon nitrogen of the Lys sidechain. In embodiments, the polymerization moiety comprises a photopolymerization moiety or a thermopolymerization moiety. In embodiments, the polymerization moiety comprises an acrylate, methacrylate, acrylamide, methacryalmide, maleimide, epoxide or fumarate group.

In embodiments, the amino acid chain or peptide chain Xaaₙ may be any sequence of Lys or Arg or a combination of Lys and Arg, from 2 to 10 amino acids in length. For the purposes of this disclosure, peptide or polypeptide is an amino acid sequence that may be chemically synthesized or made by recombinant methods. However, for the purposes of this disclosure, peptide or polypeptide is not a complete protein.

In embodiments, functionalized proteolytic enzyme-cleavable peptide monomer can be polymerized to form cell culture surfaces. In embodiments, more than one functionalized cationic peptide monomer described by Formula 1 may be combined and polymerized to form cell culture surfaces. In additional embodiments, more than one functionalized cationic peptide monomer may be combined with additional polymerizable monomers to form cell culture surfaces.

In embodiments, the polymer layer is formed from at least two different functionalized proteolytic enzyme-cleavable peptide monomers that can be described by Formula 1.

The enzyme trypsin is commonly used to release cells from cell culture surfaces. Trypsin selectively cleaves proteins at the C-terminal end of arginine and lysine. Synthetic polymeric cell culture surfaces made by polymerizing trypsin-hydrolysable monomers described by Formula 1, such as either lysine immer or arginine immer or combinations of arginine with lysine as a di-peptide dramatically accelerates the removal of cells from these surfaces. Monomers described by Formula 1 that are trypsin-cleavable include, for example, the monomers shown in Formulas 2-6:

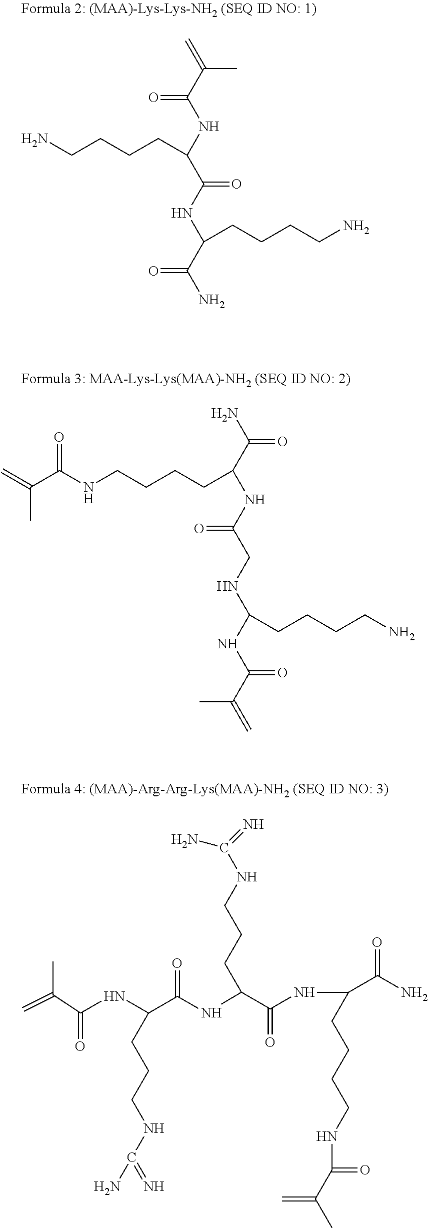

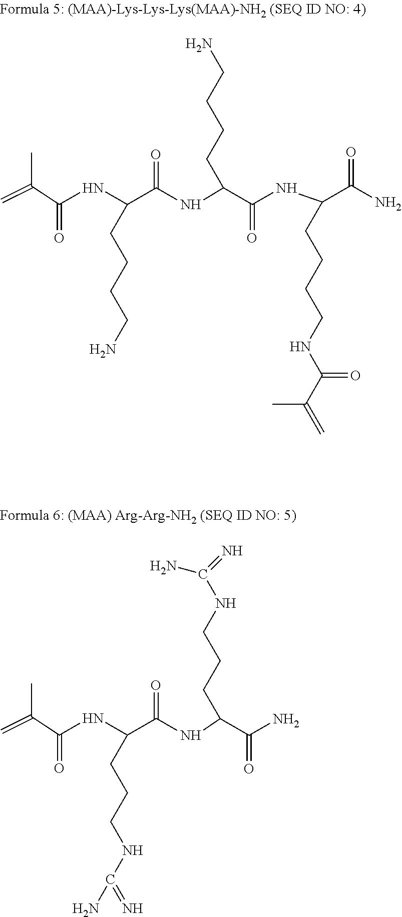

Additional monomers made from trypsin-cleavable sequences include, for example: MAA-Phe-Ala-Arg-Ile-Arg-Asp-NH₂ (SEQ ID NO:7) and MAA-Phe-Ala-Arg-Ala-Arg-Asp-NH₂ (SEQ ID NO:6). In embodiments, a polymerization moiety or polymerizable moiety may be acrylate, methacrylate, acrylamide, methacryalmide, maleimide, fumarate or epoxide group. Functionalized enzyme cleavable peptide monomers may have one or more than one polymerization moieties. If the peptide monomer has more than one polymerization moiety, the monomer is a cross-linker. In embodiments, the polymerizable moiety may be bound to the carboxyl or amino terminal of the peptide chain. In embodiments, the polymerizable moiety may be bound to a side chain of a Lys amino acid (as shown in Formula 3, 4 and 5). Enzyme-cleavable sequences are shown in Table 1, along with the enzyme that cleaves that amino acid sequence. Any of these enzyme cleavable sequences are suitable to serve as the Xaaₙ sequence.

TABLE 1

| Enzyme | Sequence | Sequence ID |
|---|---|---|
| Trypsin | Lys-Lys- | SEQ ID NO: 1 |
| Trypsin | Lys-Lys(MAA) | SEQ ID NO: 2 |
| Trypsin | Arg-Arg-Lys(MAA) | SEQ ID NO: 3 |
| Trypsin | Lys-Lys-Lys(MAA) | SEQ ID NO: 4 |
| Trypsin | Arg-Arg | SEQ ID NO: 5 |
| Trypsin | Phe-Ala-Arg-Ala-Arg-Asp | SEQ ID NO: 6 |
| Trypsin | Phe-Ala-Arg-Ile-Arg-Asp | SEQ ID NO: 7 |
| Carboxypeptidase | Ala-Tyr-Ala-Phe | SEQ ID NO: 8 |
| chymotrypsin | Ala-Leu-Phe-Ala-Leu-Arg | SEQ ID NO: 9 |
| Elastase | Ala-Ala-Ala-Ala-Leu-Phe-Arg | SEQ ID NO: 10 |
| Elastase | Ala-Ala-Pro-Ala | SEQ ID NO: 11 |
| Elastase | Ala-Ala-Pro-Val | SEQ ID NO: 12 |
| Elastase | Ala-Ala-Pro-Met | SEQ ID NO: 13 |
| Elastase | Arg-Glu-His-Val-Ile-Phe | SEQ ID NO: 14 |
| Papain | Ala-Phe-Glu-Leu-Phe-Arg | SEQ ID NO: 15 |
| Pepsin | Ala-His-Phe-Phe-Arg-Leu | SEQ ID NO: 16 |
| Plasmin | Lys-Thr-Tyr-Lys | SEQ ID NO: 17 |
| Plasmin | Lys-Thr-Phe-Lys | SEQ ID NO: 18 |
| Plasmin | Lys-Thr-Trp-Lys | SEQ ID NO: 19 |
| Plasmin | Lys-Thr-Ser-Lys | Seq ID NO: 20 |
| Plasmin | Phe-Thr-Tyr-Lys | SEQ ID NO: 21 |
| Plasmin | Leu-Thr-Phe-Lys | SEQ ID NO: 22 |
| Plasmin | Leu-Glu-Phe-Lys | SEQ ID NO: 23 |
| Thrombin | Nleu-Thr-Pro-Arg | SEQ ID NO: 24 |
| Thrombin | Leu-Thr-Pro-Arg | SEQ ID NO: 25 |
| Thrombin | Val-Thr-Pro-Arg | SEQ ID NO: 26 |
| Thrombin | Nleu-Thr-Leu-Arg | SEQ ID NO: 27 |
| Thrombin | Leu-Gly-Val-Arg | SEQ ID NO: 28 |
| Thrombin | Gly-Gly-Val-Arg | SEQ ID NO: 29 |
| Caspase | Val-Asp-Val-Ala-Asp-Val-Asp-Val-Ala-Asp | SEQ ID NO: 30 |
| Caspase | Asp-Glu-Val-Asp-Asp-Glu-Val-Asp | SEQ ID NO: 31 |
| Caspase | Ala-Ala-Ala-Asp-Ala-Ala-Asp | SEQ ID NO: 32 |
| Caspase | Leu-Glu-His-Asp-Ala-Ala-Asp | SEQ ID NO: 33 |
| Caspase | Val-Glu-Ile-Asp-Ala-Ala-Asp | SEQ ID NO: 34 |
| Caspase | Asp-Asp-Asp | SEQ ID NO: 35 |
| Collagenase | Gly-Pro-Gln-Gly-Ile-Ala-Gly-Gln | SEQ ID NO: 36 |
| Collagenase | Pro-Ser-Tyr-Phe-Leu-Asn-Ala-Gly | SEQ ID NO: 37 |
| Collagenase | Gly-Pro-Leu-Gly-Met-Arg-Gly-Leu | SEQ ID NO: 38 |
| Collagenase | Gly-Gly-Pro-Leu-Gly-Pro-Pro-Gly | SEQ ID NO: 39 |
| Cathepsin G | Leu-Leu-Ser-Ala-Leu-Gln | SEQ ID NO: 40 |
| Cathepsin G | Thr-Leu-Leu-Ser-Ala-Leu-Gln | SEQ ID NO: 41 |
| Cathepsin D | Glu-Glu-Lys-Pro-Ile-Met-Phe-Phe-Arg-Leu- | SEQ ID NO: 42 |
| | Leu-Gly-Lys-Lys | |
| Cathepsin D | Glu-Asp-Lys-Pro-Ile-Leu-Phe-Phe-Arg-Leu- | SEQ ID NO: 43 |
| | Gly-Lys | |
| Cathepsin D | Glu-Glu-Lys-Pro-Ile-Ser-Phe-Phe-Arg-Leu- | SEQ ID NO: 44 |
| | Gly-Lys | |
| Cathepsin B | Arg-Leu-Arg-Gly-Phe-Glu | SEQ ID NO: 45 |
| Cathepsin B | Arg-Ile-Ile-Glu-Gly-Ile-Glu | SEQ ID NO: 46 |

Monomers, as described by Formula 1 can be formed from the peptide cleavable sequences in Table 1 as follows: A carboxypeptidase-cleavable monomer may be, for example, (MAA)-Ala-Tyr-Ala-Phe-OH (SEQ ID NO:8). A chymotrypsin-cleavable monomer may be, for example (MAA)-Ala-Leu-Phe-Ala-Leu-Arg-NH₂ (SEQ ID NO:9). An elastase-cleavable monomer may be, for example (MAA)-Ala-Ala-Ala-Ala-Leu-Phe-Arg-NH₂ (SEQ ID NO:10), (MAA)-Ala-Ala-Pro-Ala-NH₂ (SEQ ID NO:11), (MAA)-Ala-Ala-Pro-Val-NH₂ (SEQ ID NO:12), (MAA)-Ala-Ala-Pro-Met-NH₂ (SEQ ID NO:13), or (MAA)-Arg-Glu-His-Val-Ile-Phe-NH₂ (SEQ ID NO:14). A papain-cleavable monomer may be (MAA)-Ala-Phe-Glu-Leu-Phe-Arg-NH₂ (SEQ ID NO:15). A pepsin-cleavable monomer may be (MAA)-Ala-His-Phe-Phe-Arg-Leu-NH₂ (SEQ ID NO:16) which, in embodiments, may have the L-isomer of an amino acid at position 1 or position 3, for example, L-Ala or L-Phe. A plasmin-cleavable monomer may be (MAA)-Lys-Thr-Tyr-Lys-NH₂ (SEQ ID NO:17), (MAA)-Lys-Thr-Phe-Lys-NH₂, (SEQ ID NO:18), (MAA)-Lys-Thr-Trp-Lys-NH₂ (SEQ ID NO:19), (MAA)-Lys-Thr-Ser-Lys-NH₂ (SEQ ID NO:20), (MAA)-Phe-Thr-Tyr-Lys-NH₂ (SEQ ID NO:21), (MAA)-Leu-Thr-Phe-Lys-NH₂ (SEQ ID NO:22) or (MAA)-Leu-Glu-Phe-Lys-NH₂ (SEQ ID NO:23). A thrombin-cleavable monomer may be (MAA)-Nleu-Thr-Pro-Arg-NH₂(SEQ ID NO:24), (MAA)-Leu-Thr-Pro-Arg-NH₂(SEQ ID NO:25), (MAA)-Val-Thr-Pro-Arg-NH₂ (SEQ ID NO:26), (MAA)-Nleu-Thr-Leu-Arg-NH₂ (SEQ ID NO:27) or, (MAA)-Leu-Gly-Val-Arg-NH₂ (SEQ ID NO:28) or (MAA)-Gly-Gly-Val-Arg-NH₂ (SEQ ID NO:29). A caspase-cleavable monomer may be (MAA)-Val-Asp-Val-Ala-Asp-Val-Asp-Val-Ala-Asp-NH₂, (SEQ ID NO:30), (MAA)-Asp-Glu-Val-Asp-Asp-Glu-Val-Asp-NH₂ (SEQ ID NO:31), (MAA)-Ala-Ala-Ala-Asp-Ala-Ala-Asp-NH₂ (SEQ ID NO:32), (MAA)-Leu-Glu-His-Asp-Ala-Ala-Asp-NH₂ (SEQ ID NO:33) or (MAA)-Val-Glu-Ile-Asp-Ala-Ala-Asp-NH₂ (SEQ ID NO:34) or (MAA)-Asp-Asp-Asp-NH₂ (SEQ ID NO:35). A collagenase-cleavable monomer may be (MAA)-Gly-Pro-Gln-Gly-Ile-Ala-Gly-Gln-NH₂ (SEQ ID NO:36), (MAA)-Pro-Ser-Tyr-Phe-Leu-Asn-Ala-Gly-NH₂ (SEQ ID NO:37), (MAA)-Gly-Pro-Leu-Gly-Met-Arg-Gly-Leu-NH₂ (SEQ ID NO:38), or (MAA)-Gly-Gly-Pro-Leu-Gly-Pro-Pro-Gly-NH₂ (SEQ ID NO:40). A Cathepsin G-cleavable monomer may be (MAA)-Leu-Leu-Ser-Ala-Leu-Gln-NH₂ (SEQ ID NO:40) or (MAA)-Thr-Leu-Leu-Ser-Ala-Leu-Gln-NH₂ (SEQ ID NO:41). A cathepsin D-cleavable monomer may be (MAA)-Glu-Glu-Lys-Pro-Ile-Met-Phe-Phe-Arg-Leu-Leu-Gly-Lys-Lys-NH₂ (SEQ ID NO:42), (MAA)-Glu-Asp-Lys-Pro-Ile-Leu-Phe-Phe-Arg-Leu-Gly-Lys-NH₂ (SEQ ID NO:43) or (MAA)-Glu-Glu-Lys-Pro-Ile-Ser-Phe-Phe-Arg-Leu-Gly-Lys-NH₂ (SEQ ID NO:44). A cathepsin B monomer may be (MAA)-Arg-Leu-Arg-Gly-Phe-Glu-NH₂ (SEQ ID NO:45) or (MAA)-Arg-Ile-Ile-Glu-Gly-Ile-Glu-NH₂ (SEQ ID NO:46).

The targeted hydrolytic agents may include enzymes including but not limited to; proteases, esterases, nucleases, elastases, plasmin, cathepsin, caspase and thrombin. The films may be designed such that they enable cleavage by multiple hydrolytic enzymes. For example, a film may contain a protease or nuclease susceptible collection of monomeric components. The films may also contain drug agents which when cleaved release into the media upon degradation of the biofilm by the hydrolyzing enzyme. The designed biofilms may contain 100% cleavable monomeric constituents or less than 100% cleavage sites. For example, the films may contain 50% non-hydrolyzable monomer with 50% enzymatically susceptible (hydrolysable) monomeric composition.

In embodiments, at least two functionalized enzyme-cleavable monomers are combined to form a polymer surface for cell culture. In embodiments, one of the at least two monomers is (MAA)-Lys-Lys-NH₂ (SEQ ID NO:1), (MAA)-Lys-Lys(MAA)-NH₂ (SEQ ID NO:2), (MAA)-Arg-Arg-NH₂ (SEQ ID NO:5) or (MAA)-Arg-Arg-Lys(MAA)-NH₂ (SEQ ID NO:3). In embodiments the enzyme-cleavable amino acid sequence is Lys-Lys (SEQ ID NO:1) or Arg-Arg (SEQ ID NO:5). In embodiments, additional monomers are present in a prepolymer mixture used to form an enzyme-activated polymeric cell culture surface. In embodiments, these additional monomers include, for example, methacryoyl lysine or 1-vinyl imidazole or both. The structure of 3-methacryoyl lysine is shown in Formula 7:

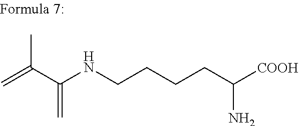

In embodiments, the enzyme cleavable cell culture surface is also made from functionalized cell adhesive peptide monomer. The functionalized cell adhesive peptide monomer may be described by Formula 8:

Z-Spₙ₂-CAP-Spₙ₃-Z¹ₙ₁ Formula 8

wherein Z and Z¹ are polymerization moieties and n1 is an integer of 0 or 1, Sp is a spacer having the formula (O—CH₂CHR′)ₘ₂ where R′ is H or CH₃ and m2 is an integer from 0 to 20, n2 is an integer of 0 or 1, n3 is an integer of 0 or 1 and CAP is a cell adhesive peptide. These polymerization moieties form the “functionalization” that allows for polymerization of the monomers. That is, a “functionalized” monomer has a polymerization moiety.

In embodiments, (in the case where no spacer is present, or where n2 and n3 both equal 0, the enzyme cleavable cell culture surface is made from a functionalized cell adhesive peptide monomer which may be described by Formula 9:

Z-CAP-Z¹ₙ₁ Formula 9

wherein Z and Z¹ are polymerization moieties and n1 is an integer of 0 or 1, wherein Z and Z¹ are polymerization moieties and n1 is an integer of 0 or 1, and CAP is a cell adhesive peptide.

In embodiments the enzyme cleavable cell culture surface comprises a cell adhesive peptide. Cell adhesive peptide sequences are shown in Table 2.

TABLE 2

| Sequence | Source |
|---|---|
| KGGGQKCIVQTTSWSQCSKS | Cyr61 res 224-240 |
| (SEQ ID NO: 47) | |
| GGGQKCIVQTTSWSQCSKS | Cyr61 res 224-240 |
| (SEQ ID NO: 48) | |
| KYGLALERKDHSG | TSP1 res 87-96 |
| (SEQ ID NO: 49) | |
| YGLALERKDHSG | TSP1 res 87-96 |
| (SEQ ID NO: 50) | |
| KGGSINNNRWHSIYITRFGNMGS | mLMα1 res 2179-2198 |
| (SEQ ID NO: 51) | |
| GGSINNNRWHSIYITRFGNMGS | mLMα1 res 2179-2198 |
| (SEQ ID NO: 52) | |
| KGGTWYKIAFQRNRK | mLMα1 res 2370-2381 |
| (SEQ ID NO: 53) | |
| GGTWYKIAFQRNRK | mLMα1 res 2370-2381 |
| (SEQ ID NO: 54) | |
| KGGTSIKIRGTYSER | mLMγ1 res 650-261 |
| (SEQ ID NO: 55) | |
| GGTSIKIRGTYSER | mLMγ1 res 650-261 |
| (SEQ ID NO: 56) | |
| KYGTDIRVTLNRLNTF | mLMγ1 res 245-257 |
| (SEQ ID NO: 57) | |
| YGTDIRVTLNRLNTF | mLMγ1 res 245-257 |
| (SEQ ID NO: 58) | |
| KYGSETTVKYIFRLHE | mLMγ1 res 615-627 |
| (SEQ ID NO: 59) | |
| YGSETTVKYIFRLHE | mLMγ1 res 615-627 |
| (SEQ ID NO: 60) | |
| KYGKAFDITYVRLKF | mLMγ1 res 139-150 |
| (SEQ ID NO: 61) | |
| YGKAFDITYVRLKF | mLMγ1 res 139-150 |
| (SEQ ID NO: 62) | |
| KYGAASIKVAVSADR | mLMα1 res2122-2132 |
| (SEQ ID NO: 63) | |
| YGAASIKVAVSADR | mLMα1 res2122-2132 |
| (SEQ ID NO: 64) | |
| KGGNGEPRGDTYRAY | BSP |
| (SEQ ID NO: 65) | |
| GGNGEPRGDTYRAY | BSP |
| (SEQ ID NO: 66) | |
| CGGNGEPRGDTRAY | BSP-Y |
| (SEQ ID NO: 67) | |
| GGNGEPRGDTRAY | BSP-Y |
| (SEQ ID NO: 68) | |
| KYGRKRLQVQLSIRT | mLMα1 res 2719-2730 |
| (SEQ ID NO: 69) | |
| YGRKRLQVQLSIRT | mLMα1 res 2719-2730 |
| (SEQ ID NO: 70) | |
| KGGRNIAEIIKDI | LMβ1 |
| (SEQ ID NO: 71) | |
| GGRNIAEIIKDI | LMβ1 |
| (SEQ ID NO: 72) | |
| KGGPQVTRGDVFTMP | VN |
| (SEQ ID NO: 73) | |
| GGPQVTRGDVFTMP | VN |
| (SEQ ID NO: 74) | |
| GGPQVTRGDVFTMPK | VN |
| SEQ ID NO: 75) | |
| GRGDSPK | Short FN |
| (SEQ ID NO: 76) | |
| KGGAVTGRGDSPASS | Long FN |
| (SEQ ID NO: 77) | |
| GGAVTGRGDSPASS | Long FN |
| (SEQ ID NO: 78) | |
| XaaPQVTRGNVFTMP | VN |
| (SEQ ID NO: 79) | |
| RGDYK | RGD |
| (SEQ ID NO: 80) | |
| GGVTRGNVFTMP | SEQ81 |
| (SEQ ID NO: 81) | |

The cell adhesive peptide may be incorporated into the polymer by mixing the functionalized cell adhesive peptide monomer, described by Formula 8, with the functionalized enzyme-cleavable monomers described by Formula 1. Or, in an alternative embodiment, cell adhesive peptide monomers may be bound to, or adsorbed to the surface of a polymer formed from functionalized enzyme cleavable monomers.

Traditionally, cationic coatings for cell culture and other life sciences related applications, including poly-L-lysine (PLL) and poly-D-lysine (PDL), have been synthesized by methods such as solid and solution phase synthesis with techniques such as Merrifield Solid Phase Synthesis. An array of protecting groups such as FMOC, t-BOC and Alloc groups are used, while carbodiimides and triazolols as activating groups have been well published. Synthesis of longer chain peptides by chemical ligation has also been used. However, these methods for synthesizing PLL and PDL surfaces can be expensive. The polymerizable-cationic monomers disclosed herein (photo- or thermal-polymerizable) provide a viable alternative to these methods for making cationic surfaces in-situ as well as polymers for coating cell culture ware because of their ability to polymerize by free radical polymerization, redox or cationic photopolymerization to form long polymer chains.

Strong interactions between cultured cells and cell culture surfaces is often desirable. For example, in cell-based drug discovery screening, scientists often culture transfected and cryopreserved division-arrested cells for use in cell-based screening assays. Strong attachment of these cells is critical to achieving a robust assay, and cell monolayer consistency is directly related to reproducible assay results.

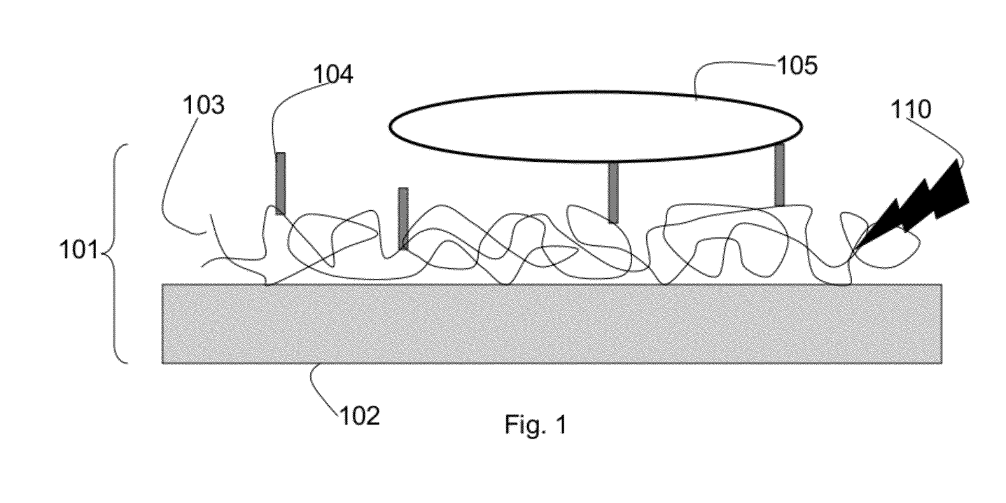

FIG. 1 is a graphic illustrating an embodiment of the cell culture surface disclosed herein. Shown is a cell culture surface 101 which includes a substrate 102, a polymer layer 103 and cell adhesive peptides 104 dispersed in the polymer layer 103. A cell 105 attaches to the cell culture surface by interacting with the cell adhesive peptides 104. Because the polymer layer 103 contains enzyme-cleavable amino acid sequences, an enzyme 110 can cleave the cell away from the cell culture surface. This can be done with minimal damage to the cell membrane and its associated proteins.

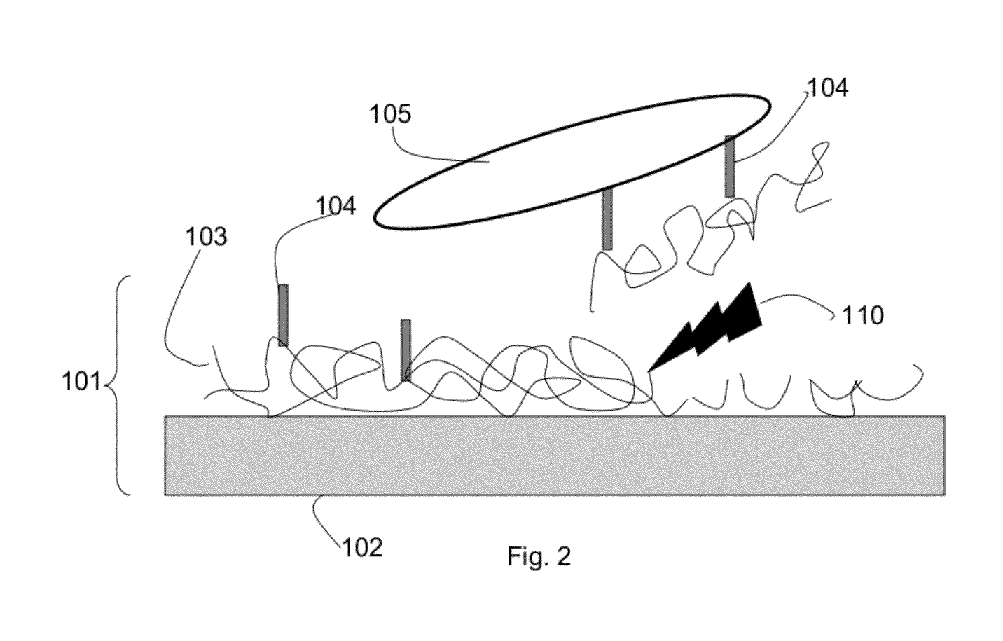

FIG. 2 is a graphic illustrating an embodiment of the cell culture surface disclosed herein. Shown is a cell culture surface 101 which includes a substrate 102, a polymer layer 103 and cell adhesive peptides 104 dispersed in the polymer layer 103. Upon enzyme treatment, the enzyme 110 cleaves the cell culture surface, releasing the cell 105. Because the polymer layer 103 contains enzyme-cleavable amino acid sequences, an enzyme 110 can cleave the cell away from the cell culture surface. This can be done with minimal damage to the cell membrane and its associated proteins.

Others have disclosed the use of a long chain PEG spacer, combined with a cell adhesive peptide sequence (an RGD sequence) and a polymerization moiety to provide a cell culture surface. For example Hern, D. L., and Hubbell, J. A., Incorporation of Adhesion Peptides into Nonadhesive Hydrogels Useful for Tissue Resurfacing, Journal of Biomedical Materials Research Part A Vol. 39, Issue 2, pp. 266-276 (Hem & Hubbell) discloses the use of cell adhesive peptides conjugated to a polymerization moiety, and the use of cell adhesive peptides conjugated to polymerization moiety via a long chain polyalkylene oxide spacer (PEG75) which was combined with PEG diacrylate (copolymerized with PEG diacrylate) to form a hydrogel cell culture surface composed primarily of PEG.

Poly-D-Lysine coatings and coated cell culture surfaces are commercially available from, for example BioOne Cell Coat® (available from Greiner, Monroe, N.C.), Bio Coat™ (available from BD, Franklin Lakes, N.J.), poly-D-lysine (Thermo Scientific, Rochester, N.Y.), poly-L-lysine (Sciencell™, Carlsbad, Calif.), and Poly-D-Lysine (Millipore, Temecula, Calif.) to name a few. In general, these coatings are made from homo-polymers of poly-lysine synthesized by solution and solid phase synthesis or fermentation. Biological sources of peptides must be purified. Commercially available cationic coatings are generally weakly physically adsorbed to surfaces to form cell culture surfaces. In embodiments of the present invention, a combination of functionalized peptide monomers are provided on a surface, and polymerized in situ. In embodiments, the surfaces are polymerized in situ using UV irradiation in the presence of a photo-initiator. This process results in polymer coatings that are strongly anchored to the surface because they are cured in-situ by free radical photo-polymerization of the cationic functionalized methacrylates to an oxygen rich thermoplastic surface. The polymeric coatings have a modulus that is tunable by varying the concentration of cationic cross-linker present in the formulation. In addition, the degree of positive charge on the surface can also be modulated by changing the concentration of different cationic species. In general, commercial offerings require careful aseptic handling. In contrast, polymeric surfaces made from monomers disclosed herein can be sterilized by any terminal sterilization method including gamma and ethylene oxide at a SAL(10⁻⁶). In addition, in general, commercial offerings for poly-D-lysine coatings are limited to 384, 96 or 6 well plates. Because the polymers of the present invention allow for strong interactions between a substrate and the polymeric coating, these surfaces are applicable in smaller and larger product formats including 1536 well plats, 384, 96, 6 well plates, as well as flasks (T25, T75, T175, T225), roller bottles, Hyperflask™ multiple layer cell culture flasks (available from Corning Incorporated, Corning, N.Y.), CellStack® or HyperStack™ (available from Corning Incorporated, Corning, N.Y.) beads and microcarriers. In embodiments, the cell culture surface may be formed on any surface suitable for cell culture. Examples of articles suitable for cell culture include single and multi-well plates, such as 6, 12, 96, 384, and 1536 well plates, jars, petri dishes, flasks, beakers, plates, roller bottles, slides, such as chambered and multichambered culture slides, tubes, cover slips, bags, membranes, hollow fibers, cups, spinner bottles, perfusion chambers, bioreactors and fermenters. In addition, embodiments of synthetic coatings prepared from the monomers disclosed herein may allow for extended shelf life and more consistency lot-to-lot and batch-to-batch. Furthermore, the technique described here in may be used on any number of substrate formats including but not limited to 2-d films, 3-D films, microplate formats, roller bottles, microcarriers or beads, biomedical substrates like stents, mat and fiber woven or non-woven surfaces.

Examples of embodiments of the mixtures of functionalized cationic peptide monomers used to form cell culture surfaces of the present invention are presented in Tables 3-4.

TABLE 3

| Formulation | PARG-1 |
|---|---|
| MAA-Arg-Arg-NH2 (SEQ ID NO: 5) | 400 |
| (μl) | |
| MAA-Lys-Lys(MAA)-NH2 (SEQ ID NO: 2) | 400 |
| (μl) | |
| 1-Vinyimidazole | 200 |
| 3-Methacryloyl-(L)-Lysine (μl) | 200 |
| Darocur 1173 (10% in EtOH) (μl) | 15 |
| Irgacure 1-819 (1% in EtOH) (μl) | 50 |
| Ethanol (ml) | 8.8 |
| VN-Methacrylate | 425 mg |

TABLE 4

| Formulation | PLYS-1 |
|---|---|
| MAA-Lys-Lys-NH2 (SEQ ID NO: 1) | 400 |
| (μl) | |
| MAA-Lys-Lys(MAA)-NH2 (SEQ ID NO: 2) | 400 |
| (μl) | |
| 1-Vinyimidazole | 200 |
| 3-Methacryloyl-(L)-Lysine (μl) | 200 |
| Darocur 1173 (10% in EtOH) (μl) | 15 |
| Irgacure 1-819 (1% in EtOH) (μl) | 50 |
| Ethanol (ml) | 8.8 |
| VN-Methacrylate | 425 mg |

VN-methacrylate refers to a vitronectin sequence (SEQ ID NO:74) or (SEQ ID NO:79, for example) having a methacrylate group on the carboxy terminal of the amino acid sequence. In embodiments, the VN-methacrylate monomer is amidated at the amino terminal end of the amino acid sequence. Some embodiments, including the embodiments shown in Tables 3-4 include 1-vinyl imidazole and methacryoyl lysine. Vinyl imidazole is a cationic five member ring monomer that is used to confer additional positive charge to the surface. Methacryoyl lysine is a zwitterionic monomer that is used to provide compatibility and matching reaction rates to di-lysine (SEQ ID NO: 1) and di-arginine (SEQ ID NO: 5) methacrylate monomer. Some embodiments do not include these monomers. Embodiments without vinyl imidazole and methacryoyl lysine provided useful cell culture surfaces. Not wishing to be constrained by theory, the addition of the components vinyl imidazole and methacryoyl lysine may also serve to provide a base matrix that provides for weak or limited interaction with the cells own extracellular matrix and thereby also allowing for facile enzymatic digestion of cells from the surface. Additional zwitterionic agenst such as sulfobetaines may also provide similar non-stick capability. Additional examples may include [2-(methacryloyloxy) ethyl]dimethyl-(3-sulfopropyl) ammonium hydroxide or [3-(Methacryoylamino)propyl]-dimethyl(3-sulfopropyl) ammonium hydroxide. However, surfaces prepared without functionalized cationic peptide monomers were not useful cell culture surfaces (data not shown).

In embodiments, the formulations shown in Tables 1-4 also include photoinitiators and solvents. For example, in each of the formulations shown in Tables 1-4, 15 μl of Darocur 1173 (10% in Ethanol), 50 μl of Irgacure I-819 (1% in ethanol) and 8.8 ml of ethanol were present in the formulations, to coat a well of a 96 well plate. Mote that in some formulations a 200 proof solution of ethanol is not exclusively required and other solvents or adjusted solvents may also be used or substituted to aid the solubility of the monomeric components. Alternative solvent substitutions may include but are not limited to: 70% ethanol with 30% water or 69% ethanol, 1% DMSO and 30% water, and so on. In embodiments, the methods of making peptide-containing polymeric cell culture surfaces provide (1) coating functionalized cationic peptide monomers, or mixtures thereof on a substrate; (2) curing or polymerizing the monomers to form polymers; (3) washing; (4) drying; and, (5) sterilizing. In embodiments, additional steps may include packaging and/or shipping the cell culture article having a cationic polymeric cell culture surface made from functionalized cationic peptide monomers.

Depending upon the formulation, and the amount of monomer used in the preparation of the cell culture coating, the polymeric coating may exhibit variable thicknesses. For example, in embodiments, the thickness of polymeric coatings ranged from approximately 1 nm to approximately 200 nm.

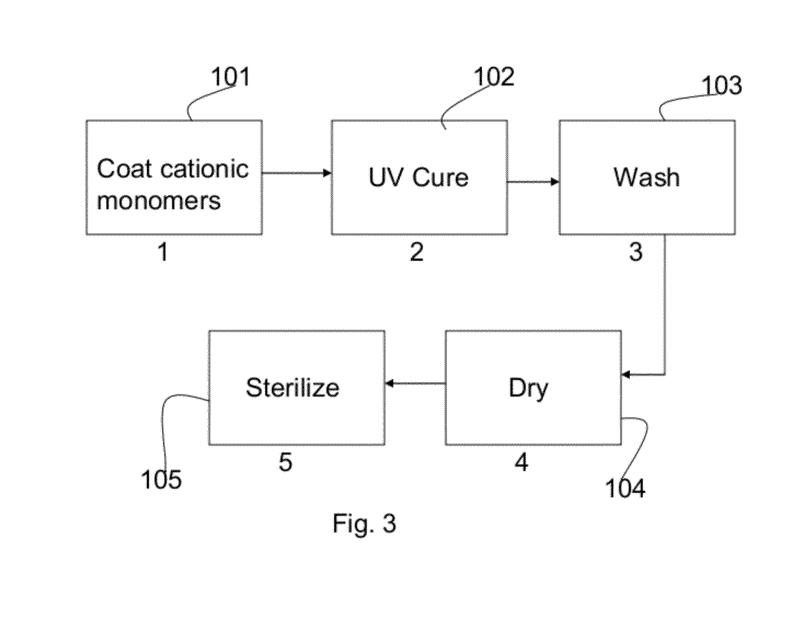

FIG. 3 is a flow chart showing an embodiment of a method of making cell culture surfaces. In embodiments, methods for forming polymeric cationic cell culture surfaces by providing functionalized cationic peptide monomers, to the surface of a substrate and polymerizing the monomers to form a polymeric surface are provided. These methods include steps of (101) applying a solution of monomers containing functionalized enzyme cleavable peptide monomers, with cell adhesive peptide monomers and, optionally with additional “base matrix” (meaning monomers such as 3-methacryoyl lysine and 1-vinyl imidazole), to a cell culture substrate; (102) polymerize the monomers and the functionalized peptide by, for example, exposure to UV/VIS energy to cure the monomers; (103) wash; (104) dry; (105) sterilize. Optional additional treatments include applying a top to a topless flask (welding the top to the flask, for example), labeling, packaging and shipping. In step (101) the mixture of functionalized cationic peptide monomer may be provided to the surface of a substrate by any means know in the art including liquid dispensing, spin coating, spray coating, or other methods. In step (102), the curing or polymerizing step may be accomplished by any means known in the art, and depending upon the nature of the polymerizing moiety, and may include the introduction of photoinitiators into the monomer mixture and the exposure of the surface to UV, visible or thermal energy. In step (103) washing may be accomplished by any means known in the art including liquid dispensing and incubating, with or without agitation, where the liquid may be water, a lower alcohol, a lower alcohol diluted in water, or other solvent. In step (104), the drying step may be present or absent, and may be accomplished by the application of a vacuum and/or heat. In step (105), sterilization may occur by exposure to ethanol, for example, gamma irradiation, or other methods.

In embodiments, in step 101, addition to monomers, a composition forming the layer may include one or more additional compounds such as surfactants, wetting agents, photoinitiators, chain transfer agents, thermal initiators, catalysts additional monomers and activators. Any suitable polymerization initiator may be employed. One of skill in the art will readily be able to select a suitable initiator, e.g. a radical initiator or a cationic initiator, suitable for use with the monomers. In various embodiments, UV light is used to generate free radical monomers to initiate chain polymerization. However, visible light initiators and low temperature initiators may be used instead of UV initiators to shield the peptide from exposure to a more harmful or damaging radiation source such as UV radiation.

Any suitable initiator may be used, including thermal initiators, photo-initiators or room temperature initiators. Examples of polymerization initiators include organic peroxides, azo compounds, quinones, nitroso compounds, acyl halides, hydrazones, mercapto compounds, pyrylium compounds, imidazoles, chlorotriazines, benzoin, benzoin alkyl ethers, diketones, phenones, or mixtures thereof. Potassium persulfate may be used as an initiator for room temperature polymerization. Examples of suitable commercially available, ultraviolet-activated and visible light-activated photoinitiators have tradenames such as IRGACURE 651, IRGACURE 184, IRGACURE 369, IRGACURE 819, DAROCUR 4265 and DAROCUR 1173 commercially available from Ciba Specialty Chemicals, Tarrytown, N.Y. and LUCIRIN TPO and LUCIRIN TPO-L commercially available from BASF (Charlotte, N.C.)

Additional initiators may include water soluble azo-initiators that can be used in thermal polymerization including, for example, (VA-044) 2,2′-Azobis[2-(2-imidazolin-2-yl)propane]dihydrochloride; (VA 046B) 2,2′-Azobis[2-(2-imidazolin-2-yl)propane]disulfate dehydrate; (VA-50) 2,2′-Azobis (2-methylpropionamidine)dihydrochloride; (VA-057) 2,2′-Azobis[N-(2-carboxyethyl)-2-methylpropionamidine]hydrate; (VA-060) 2,2′-Azobis{2-[1-(2-hydroxyethyl)-2-imidazolin-2-yl]propane}dihydrochloride; (VA-061) 2,2′-Azobis[2-(2-imidazolin-2-yl)propane]; (VA-067) 2,2′-Azobis(1-imino-1-pyrrolidino-2-ethylpropane)dihydrochloride; (VA-080) 2,2′-Azobis{2-methyl-N-[1,1-bis(hydroxymethyl)-2-hydroxyethyl]propionamide or (VA-086) 2,2′-Azobis[2-methyl-N-(2-hydroxyethyl)propionamide]. Oil soluble azo-initiators such as (V-70) 2,2′-Azobis(4-methoxy-2.4-dimethyl valeronitrile); (V-65) 2,2′-Azobis(2.4-dimethyl valeronitrile); (V-601) Dimethyl 2,2′-azobis(2-methylpropionate); (V-59) 2,2′-Azobis(2-methylbutyronitrile; (V-40) 1,1′-Azobis(cyclohexane-1-carbonitrile); (VF-096) 2,2′-Azobis[N-(2-propenyl)-2-methylpropionamide]; (V-30) 1-[(1-cyano-1-methylethyl)azo]formamide; (VAm-110) 2,2′-Azobis(N-butyl-2-methylpropionamide) or (VAm-111) 2,2′-Azobis(N-cyclohexyl-2-methylpropionamide) may also be used in thermal polymerization. These initiators are available from for example, WAKO Chemicals, Richmond Va. In addition, macro-initiators, such as azo-initiators having a PEG backbone may be used in thermal polymerization.

A photosensitizer may also be included in a suitable initiator system. Representative photosensitizers have carbonyl groups or tertiary amino groups or mixtures thereof. Photosensitizers having a carbonyl groups include benzophenone, acetophenone, benzil, benzaldehyde, o-chlorobenzaldehyde, xanthone, thioxanthone, 9,10-anthraquinone, and other aromatic ketones. Photosensitizers having tertiary amines include methyldiethanolamine, ethyldiethanolamine, triethanolamine, phenylmethyl-ethanolamine, and dimethylaminoethylbenzoate. Commercially available photosensitizers include QUANTICURE ITX, QUANTICURE QTX, QUANTICURE PTX, QUANTICURE EPD from Biddle Sawyer Corp., Crawley, England.

In general, the amount of photosensitizer or photoinitiator system may vary from about 0.01 to 10% by weight.

Examples of cationic initiators include salts of onium cations, such as arylsulfonium salts, as well as organometallic salts such as ion arene systems.

In embodiments, the substrate may be any material suitable for culturing cells, including a ceramic substance, a glass, a plastic, a polymer or co-polymer, any combinations thereof, or a coating of one material on another. The substrate may be flat or shaped. Such substrates include glass materials such as soda-lime glass, pyrex glass, vycor glass, quartz glass; silicon; plastics or polymers, including dendritic polymers, such as poly(vinyl chloride), poly(vinyl alcohol), poly(methyl methacrylate), poly(vinyl acetate-co-maleic anhydride), poly(dimethylsiloxane) monomethacrylate, cyclic olefin polymers, fluorocarbon polymers, polystyrenes, polypropylene, polyethyleneimine; copolymers such as poly(vinyl acetate-co-maleic anhydride), poly(styrene-co-maleic anhydride), poly(ethylene-co-acrylic acid) or derivatives of these or the like. As used herein, “cyclic olefin copolymer” means a polymer formed from more than one monomer species, where at least one of the monomer species is a cyclic olefin monomer and at least one other monomer species is not a cyclic olefin monomer species. In many embodiments, cyclic olefin copolymers are formed from ethylene and norbonene monomers. Cyclic olefin copolymer resins are commercially available with trade name of TOPAS® from Boedeker Plastics, Inc., Japan and Zeonor from Zeon Chemicals, L.P. Lousiville, Ky. In embodiments, the substrate may be treated to enhance retention of the polymer matrix. For example, the substrate may be treated with chemical or plasma treatments which provide negative charge, positive charge, create a more hydrophilic surface, or create functional chemical groups that enhance the adhesion of the polymer matrix to the substrate. For example, such treatments may include hydrophobic or hydrophilic interactions, steric interactions, affinities or Vander Waal forces.

To form the enzyme-cleavable synthetic cell culture surface, the monomers are polymerized. Whether polymerized in bulk phase (substantially solvent free) or solvent phase, the monomers are polymerized via an appropriate initiation mechanism. Many such mechanisms are known in the art. For example, temperature may be increased to activate a thermal initiator; photoinitiators may be activated by exposure to appropriate wavelength of light, or the like. According to numerous embodiments, the monomer or monomer mixture is cured using UV light. The curing preferably occurs under inert gas protection, such as nitrogen protection, to prevent oxygen inhibition. Suitable UV light combined with gas protection may increase polymer conversion, insure coating integrity and reduce cytotoxicity.

In embodiments, the layer may be washed with solvent one or more times to remove impurities such as unreacted monomers, residual photo-initiators or initiators, or low molecular weight polymer species. In various embodiments, the layer is washed with ethanol or an ethanol-water solution, e.g. 70% ethanol, greater than 90% ethanol, greater than 95% ethanol or greater than about 99% ethanol. Washing with a 70% ethanol solvent may not only serve to remove impurities, which may be cytotoxic, but also can serve to sterilize the surface prior to incubation with cells.

Human embryonic mesenchymal cells were grown on embodiments of the enzyme cleavable polymeric surface. Cells tested included a human embryonic kidney cell line HEK-293 (ATCC#CRL-1573), a human hepatocellular carcinoma cell line HEP-G2 (ATCC#HB-8065), a mouse neuroblastoma cell line (Neuro-2a (ATCC#CCL-131) and a rat pheochromocytoma (adrenal cell) cell line PC-12 (ATCC#CRL-1721. Cells were grown in the presence of Mediatech IMDM (available from Mediatech, Manassas, Va.) media supplemented with 10% or 2% fetal bovine serum (FBS, available from Mediatech).

Cells may be used for any suitable purpose, including (i) for investigational studies of the cells in culture, (iii) for developing therapeutics including therapeutic cells, (v) for studying gene expression, e.g. by creating cDNA libraries, and (vi) for studying drug interactions with cells and toxicity screening.

Cell culture articles prepared according to embodiments of the methods of the present invention can be effectively presented to facilitate growth and proliferation of any relevant cell type, including, primary cells, cell lines, tissues and, for example, stem cells, adult stem cells, Embryonic Stem Cells (ESCs), human Embryonic Stem Cells (hESCs), human mesenchymal stem cells (hMSC) or Inducible Pluripotent cells (IPCs). In embodiments, these cells in culture may be used in therapeutic applications. IPCs according to the invention may also be differentiated from induced primate pluripotent stem (iPS) cells. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst as well as surface antigen expression, gene expression and telomerase activity resembling blastocyst derived embryonic stem cells. iPS cells typically have the ability to differentiate into at least one cell type from each of the primary germ layers: ectoderm, endoderm and mesoderm and thus are suitable for differentiation into a variety of cell types. The iPS cells, like hESC, also form teratomas when injected into immuno-deficient mice, e.g., SCID mice. (Takahashi et al., (2007) Cell 131(5):861; Yu et al., (2007) Science 318:5858).

Advantages of the synthetic polymeric cell culture surfaces prepared using the functionalized cationic peptide monomers disclosed herein include that surfaces promote the binding of weakly adherent cells, the surfaces can be sterilized using gamma sterilization methods, may exhibit extended stability and shelf life, the coatings are transparent, and are compatible with fluorescent and colorimetric assays. In addition, the surfaces are entirely synthetic. In addition, the coatings are prepared from environmentally friendly starting materials.

In an aspect (1), an apparatus for cell culture is provided comprising: a substrate; a polymer layer on the substrate, the polymer layer comprising a proteolytic enzyme-cleavable amino acid sequence; and a cell adhesive amino acid sequence. In an aspect (2) the apparatus of aspect 1 is provided wherein the polymer layer is formed from at least two functionalized enzyme-cleavable peptide monomers of the formula: Z-Xaaₙ-Z¹ₙ₁ wherein Z and Z¹ are polymerization moieties and n1 is an integer of 0 or 1, wherein Xaa is each independently an amino acid and n is an integer from 2 to 100, and wherein at least one Xaaₙ is an amino acid sequence that is cleavable by a protease enzyme; and a functionalized cell adhesive peptide monomer of the formula: Z-CAP-Z¹ₙ₁ wherein Z and Z¹ are polymerization moieties and n1 is an integer of 0 or 1, and CAP is a cell adhesive peptide. In an aspect (3), the apparatus of aspect 1 or 2 is provided wherein Xaaₙ comprises at least one Lys amino acid, and at least one Lys amino acid of Xaaₙ comprises a polymerization moiety at the epsilon nitrogen of the Lys sidechain. In an aspect (4), the apparatus of aspects 1 or 2 is provided wherein the polymerization moiety comprises an acrylate, methacrylate, acrylamide, vinyl, methacryalmide, maleimide, epoxide or fumarate group. In an aspect (5) the apparatus of any one of aspects 1-4 is provided wherein the functionalized enzyme-cleavable peptide monomer comprises a trypsin-cleavable sequence. In an aspect (6), the apparatus of any one of aspects 1-5 is provided wherein one of the at least two monomers is MAA-Lys-Lys-NH₂ (SEQ ID NO:1). In an aspect (7) the apparatus of any one of aspects 1-5 is provided wherein one of the at least two monomers is MAA-Lys-Lys(MAA)-NH₂ (SEQ ID NO:2). In an aspect (8), the apparatus of any one of aspects 1, 2, 4 or 5 is provided wherein one of the at least two monomers is MAA-Arg-Arg-NH₂ (SEQ ID NO:5). In an aspect (9), the apparatus of any one of aspects 1-5 is provided wherein one of the at least two monomers is MAA-Arg-Arg-Lys(MAA)-NH₂ (SEQ ID NO:3). In an aspect (10) the apparatus of any one of aspects 1, 2, 4 or 5 is provided wherein the enzyme-cleavable amino acid sequence is Lys-Lys (SEQ ID NO: 1) or Arg-Arg (SEQ ID NO: 5). In an aspect (11), the apparatus of aspect 1 or 2 is provided wherein the functionalized enzyme-cleavable peptide monomer comprises a carboxypeptidase-cleavable sequence. In an aspect (12) the apparatus of aspect 11 is provided wherein at least one of the at least two monomers comprises (MAA)-Ala-Tyr-Ala-Phe-OH (SEQ ID NO:8). In an aspect (13), the apparatus of aspect 1 or 2 is provided wherein the functionalized enzyme-cleavable peptide monomer comprises a chymotrypsin-cleavable sequence. In an aspect (14) the apparatus of aspect 13 is provided wherein the functionalized enzyme-cleavable peptide monomer comprises (MAA)-Ala-Leu-Phe-Ala-Leu-Arg-NH₂ (SEQ ID NO: 9). In an aspect (15) the apparatus of aspect 1 or 2 is provided wherein the functionalized enzyme-cleavable peptide monomer comprises an elastase-cleavable sequence. In an aspect (16) the apparatus of aspect 15 is provided wherein the functionalized enzyme-cleavable peptide monomer comprises (MAA)-Ala-Ala-Ala-Ala-Leu-Phe-Arg-NH₂ (SEQ ID NO: 10), (MAA)-Ala-Ala-Pro-Ala-NH₂ (SEQ ID NO: 11), (MAA)-Ala-Ala-Pro-Val-NH₂ (SEQ ID NO: 12), (MAA)-Ala-Ala-Pro-Met-NH₂ (SEQ ID NO: 13) or (MAA)-Arg-Glu-His-Val-Ile-Phe-NH₂ (SEQ ID NO: 14). In an aspect (17), the apparatus of aspect 1 or 2 is provided wherein the functionalized enzyme-cleavable peptide monomer comprises a papain-cleavable sequence. In an aspect (18), the apparatus of aspect 17 is provided wherein the functionalized enzyme-cleavable peptide monomer comprises (MAA)-Ala-Phe-Glu-Leu-Phe-Arg-NH₂ (SEQ ID NO: 15). In an aspect (19) the apparatus of aspect 1 or 2 is provided wherein the functionalized enzyme-cleavable peptide monomer comprises a pepsin-cleavable sequence. In an aspect (20) the apparatus of aspect 19 is provided wherein the functionalized enzyme-cleavable peptide monomer comprises (MAA)-Ala-His-Phe-Phe-Arg-Leu-NH₂ (SEQ ID NO: 16) where the first Alanine amino acid may be the L-isomer, or (MAA)-Ala-His-Phe-Phe-Arg-Leu-NH₂ (SEQ ID NO: 16). In an aspect (21) the apparatus of aspect 1 or 2 is provided wherein the functionalized enzyme-cleavable peptide monomer comprises a plasmin-cleavable sequence. IN an aspect (22), the apparatus of aspect 21 is provided wherein the functionalized enzyme-cleavable peptide monomer comprises (MAA)-Lys-Thr-Tyr-Lys-NH₂ (SEQ ID NO: 17), (MAA)-Lys-Thr-Phe-Lys-NH₂ (SEQ ID NO: 18), (MAA)-Lys-Thr-Trp-Lys-NH₂ (SEQ ID NO: 19), (MAA)-Lys-Thr-Ser-Lys-NH₂ (SEQ ID NO: 20), (MAA)-Phe-Thr-Tyr-Lys-NH₂ (SEQ ID NO: 21), (MAA)-Leu-Thr-Phe-Lys-NH₂ (SEQ ID NO: 22), or (MAA)-Leu-Glu-Phe-Lys-NH₂ (SEQ ID NO: 23). In an aspect (23), the apparatus of aspect 1 or 2 is provided wherein the functionalized enzyme-cleavable peptide monomer comprises a thrombin-cleavable sequence. In an aspect (24) the apparatus of aspect 23 is provided wherein the functionalized enzyme-cleavable peptide monomer comprises (MAA)-Nleu-Thr-Pro-Arg-NH₂ (SEQ ID NO: 24), (MAA)-Val-Thr-Pro-Arg-NH₂ (SEQ ID NO: 26), (MAA)-Nleu-Thr-Leu-Arg-NH₂ (SEQ ID NO: 27), (MAA)-Leu-Gly-Val-Arg-NH₂ (SEQ ID NO: 28) or (MAA)-Gly-Gly-Val-Arg-NH₂ (SEQ ID NO: 29). In an aspect (25) the apparatus of aspect 1 or 2 is provided wherein the functionalized enzyme-cleavable peptide monomer comprises a caspase-cleavable sequence. In an aspect (26), the apparatus of aspect 25 is provided wherein the functionalized enzyme-cleavable peptide monomer comprises (MAA)-Val-Asp-Val-Ala-Asp-Val-Asp-Val-Ala-Asp-NH₂ (SEQ ID NO: 30), (MAA)-Asp-Glu-Val-Asp-Asp-Glu-Val-Asp-NH₂ (SEQ ID NO: 31), (MAA)-Ala-Ala-Ala-Asp-Ala-Ala-Asp-NH₂ (SEQ ID NO: 32), (MAA)-Leu-Glu-His-Asp-Ala-Ala-Asp-NH₂ (SEQ ID NO: 33), (MAA)-Val-Glu-Ile-Asp-Ala-Ala-Asp-NH₂ (SEQ ID NO: 34) or (MAA)-Asp-Asp-Asp-NH₂ (SEQ ID NO: 35). IN an aspect (27) the apparatus of aspect 1 or 2 is provided wherein the functionalized enzyme-cleavable peptide monomer comprises a collagenase-cleavable sequence. In an aspect (28) the apparatus of aspect 27 is provided wherein the functionalized enzyme-cleavable peptide monomer comprises (MAA)-Gly-Pro-Gln-Gly-Ile-Ala-Gly-Gln-NH₂ (SEQ ID NO: 36), (MAA)-Pro-Ser-Tyr-Phe-Leu-Asn-Ala-Gly-NH₂ (SEQ ID NO: 37), (MAA)-Gly-Pro-Leu-Gly-Met-Arg-Gly-Leu-NH₂ (SEQ ID NO: 38), or (MAA)-Gly-Gly-Pro-Leu-Gly-Pro-Pro-Gly-NH₂ (SEQ ID NO: 39). In an aspect (29) the apparatus of aspect 1 or 2 is provided wherein the functionalized enzyme-cleavable peptide monomer comprises a cathepsin G-cleavable sequence. In an aspect, the apparatus of aspect 29 is provided wherein the functionalized enzyme-cleavable peptide monomer comprises (MAA)-Leu-Leu-Ser-Ala-Leu-Gln-NH₂ (SEQ ID NO: 40) or (MAA)-Thr-Leu-Leu-Ser-Ala-Leu-Gln-NH₂ (SEQ ID NO: 41). In an aspect (30), the apparatus of aspect 1 or 2 is provided wherein the functionalized enzyme-cleavable peptide monomer comprises a cathepsin D-cleavable sequence. In an aspect (31) the apparatus of aspect 31 is provided wherein the functionalized enzyme-cleavable peptide monomer comprises (MAA)-Glu-Glu-Lys-Pro-Ile-Met-Phe-Phe-Arg-Leu-Leu-Gly-Lys-Lys-NH₂ (SEQ ID NO: 42), (MAA)-Glu-Asp-Lys-Pro-Ile-Leu-Phe-Phe-Arg-Leu-Gly-Lys-NH₂ (SEQ ID NO: 43) or (MAA)-Glu-Glu-Lys-Pro-Ile-Ser-Phe-Phe-Arg-Leu-Gly-Lys-NH₂ (SEQ ID NO: 44). IN an aspect (32) the apparatus of aspect 1 or 2 is provided wherein the functionalized enzyme-cleavable peptide monomer comprises Cathepsin B cleavable sequence. In an aspect (33) the apparatus of aspect 33 is provided wherein the functionalized enzyme-cleavable peptide monomer comprises (MAA)-Arg-Leu-Arg-Gly-Phe-Glu-NH₂ (SEQ ID NO: 45), or (MAA)-Arg-Ile-Ile-Glu-Gly-Ile-Glu-NH₂ (SEQ ID NO: 46). In an aspect (34) the apparatus of any one of aspects 1-34 is provided wherein the cell adhesive peptide is selected from the group consisting of KGGGQKCIVQTTSWSQCSKS (SEQ ID NO:47); GGGQKCIVQTTSWSQCSKS(SEQ ID NO:48); KYGLALERKDHSG (SEQ ID NO:49); YGLALERKDHSG (SEQ ID NO:50); KGGSINNNRHSIYITRFGNMGS (SEQ ID NO:51); GGSINNNRHSIYITRFGNMGS (SEQ ID NO:52); KGGTWYKIAFQRNRK (SEQ ID NO:53); GGTWYKIAFQRNRK (SEQ ID NO:54); KGGTSIKIRGTYSER (SEQ ID NO:55); GGTSIKIRGTYSER (SEQ ID NO:56); KYGTDIRVTLNRLNTF (SEQ ID NO:57); YGTDIRVTLNRLNTF (SEQ ID NO:58); KYGSETTVKYIFRLHE (SEQ ID NO:59); YGSETTVKYIFRLHE(SEQ ID NO:60); KYGKAFDITYVRLKF (SEQ ID NO:61); YGKAFDITYVRLKF(SEQ ID NO:62); KYGAASIKVAVSADR (SEQ ID NO:63); YGAASIKVAVSADR(SEQ ID NO:64); KGGNGEPRGDTYRAY(SEQ ID NO:65); GGNGEPRGDTYRAY (SEQ ID NO:66) CGGNGEPRGDTRAY (SEQ ID NO:67); GGNGEPRGDTRAY (SEQ ID NO:68); KYGRKRLQVQLSIRT (SEQ ID NO:69); YGRKRLQVQLSIRT(SEQ ID NO:70); KGGRNIAEIIKDI (SEQ ID NO:71); GGRNIAEIIKDI (SEQ ID NO:72); KGGPQVTRGDVFTMP (SEQ ID NO:73); GGPQVTRGDVFTMP(SEQ ID NO:74); GGPQVTRGDVFTMPK (SEQ ID NO:75); GRGDSPK (SEQ ID NO:76); KGGAVTGRGDSPASS(SEQ ID NO:77); GGAVTGRGDSPASS (SEQ ID NO:78); XaaPQVTRGNVFTMP (SEQ ID NO:79); RGDYK (SEQ ID NO:80), where the peptide sequences may be linear or cyclic, or combinations. In an aspect (35) the apparatus of any one of aspects 1-35 is provided further comprising 3-methacryoyl lysine. In an aspect (36) the apparatus of any one of aspects 1-36 is provided further comprising 1-vinyl imidazole. In an aspect (37) the apparatus of any one of aspects 1-37 is provided wherein the substrate comprises ceramic, glass, plastic, a polymer or co-polymer, any combinations thereof, or a coating of one material on another.

In an additional aspect (38) An apparatus for cell culture is provided, comprising: a substrate; a polymer layer on the substrate, the polymer layer formed from a functionalized proteolytic enzyme-cleavable peptide monomer, a functionalized cell adhesive peptide monomer, 3-methacryoyl lysine and 1-vinyl imidazole; wherein the functionalized proteolytic enzyme-cleavable peptide monomer has the formula: Z-Xaaₙ-Z¹ₙ₁, wherein Z and Z¹ are polymerization moieties and n1 is an integer of 0 or 1, wherein Xaa is each independently any amino acid and n is an integer from 2 to 100, and wherein at least one Xaaₙ is an amino acid sequence that is cleavable by a protease enzyme; and wherein the functionalized cell adhesive peptide monomer has the formula: Z-CAP-Z¹ₙ₁ wherein Z and Z¹ are polymerization moieties and n1 is an integer of 0 or 1, and CAP is a cell adhesive peptide. In an aspect (39) apparatus of aspect 38 is provided wherein the substrate comprises ceramic, glass, plastic, a polymer or co-polymer, any combinations thereof, or a coating of one material on another. In an aspect (40) the apparatus of aspect 38 is provided wherein Xaaₙ comprises at least one Lys amino acid, and at least one Lys amino acid of Xaaₙ comprises a polymerization moiety at the epsilon nitrogen of the Lys sidechain. In an aspect (41) the apparatus of aspect 38 is provided wherein the polymerization moiety comprises an acrylate, methacrylate, vinyl, acrylamide, methacryalmide, maleimide, epoxide or fumarate group. In an aspect (42) the apparatus of aspect 38 is provided wherein the functionalized enzyme-cleavable peptide monomer comprises a trypsin-cleavable sequence. In an aspect (42) the apparatus of aspect 38 is provided wherein one of the at least two monomers is Lys-Lys-NH₂ (SEQ ID NO: 1) wherein at least one of the Lys amino acids has a methacrylate group (MAA) at the epsilon nitrogen of the Lys sidechain. In an aspect (43) the apparatus of aspect 38 is provided wherein the proteolytic enzyme-cleavable peptide monomer comprises MAA-Arg-Arg-NH₂ (SEQ ID NO: 5). In an aspect (44) the apparatus of aspect 38 is provided wherein the proteolytic enzyme-cleavable peptide monomer comprises MAA-Arg-Arg-Lys(MAA)-NH₂(SEQ ID NO: 3). In an aspect (45) the apparatus of aspect 38 is provided wherein the functionalized enzyme-cleavable peptide monomer comprises a carboxypeptidase-cleavable sequence. In an aspect (46) the apparatus of aspect 38 is provided wherein the functionalized enzyme-cleavable peptide monomer comprises a chymotrypsin-cleavable sequence. In an aspect (47) the apparatus of aspect 38 is provided wherein the functionalized enzyme-cleavable peptide monomer comprises an elastase-cleavable sequence. In an aspect (48) the apparatus of aspect 38 is provided wherein the functionalized enzyme-cleavable peptide monomer comprises a papain-cleavable sequence. In an aspect (49) the apparatus of aspect 38 is provided wherein the functionalized enzyme-cleavable peptide monomer comprises a pepsin-cleavable sequence. In an aspect (50) the apparatus of aspect 38 is provided wherein the functionalized enzyme-cleavable peptide monomer comprises a plasmin-cleavable sequence. In an aspect (51) the apparatus of aspect 38 is provided wherein the functionalized enzyme-cleavable peptide monomer comprises a thrombin-cleavable sequence. In an aspect (52) the apparatus of aspect 38 is provided wherein the functionalized enzyme-cleavable peptide monomer comprises a caspase-cleavable sequence. In an aspect (53) the apparatus of aspect 38 is provided wherein the functionalized enzyme-cleavable peptide monomer comprises a collagenase-cleavable sequence. In an aspect (54) the apparatus of aspect 38 is provided wherein the functionalized enzyme-cleavable peptide monomer comprises a cathepsin G-, cathepsin B or cathepsin D-cleavable sequence. In an aspect (55) the apparatus of aspect 38 is provided wherein the cell adhesive peptide is selected from the group consisting of KGGGQKCIVQTTSWSQCSKS (SEQ ID NO:47); GGGQKCIVQTTSWSQCSKS(SEQ ID NO:48); KYGLALERKDHSG (SEQ ID NO:49); YGLALERKDHSG (SEQ ID NO:50); KGGSINNNRHSIYITRFGNMGS (SEQ ID NO:51); GGSINNNRHSIYITRFGNMGS (SEQ ID NO:52); KGGTWYKIAFQRNRK (SEQ ID NO:53); GGTWYKIAFQRNRK (SEQ ID NO:54); KGGTSIKIRGTYSER (SEQ ID NO:55); GGTSIKIRGTYSER (SEQ ID NO:56); KYGTDIRVTLNRLNTF (SEQ ID NO:57); YGTDIRVTLNRLNTF (SEQ ID NO:58); KYGSETTVKYIFRLHE (SEQ ID NO:59); YGSETTVKYIFRLHE(SEQ ID NO:60); KYGKAFDITYVRLKF (SEQ ID NO:61); YGKAFDITYVRLKF(SEQ ID NO:62); KYGAASIKVAVSADR (SEQ ID NO:63); YGAASIKVAVSADR(SEQ ID NO:64); KGGNGEPRGDTYRAY(SEQ ID NO:65); GGNGEPRGDTYRAY (SEQ ID NO:66) CGGNGEPRGDTRAY (SEQ ID NO:67); GGNGEPRGDTRAY (SEQ ID NO:68); KYGRKRLQVQLSIRT (SEQ ID NO:69); YGRKRLQVQLSIRT(SEQ ID NO:70); KGGRNIAEIIKDI (SEQ ID NO:71); GGRNIAEIIKDI (SEQ ID NO:72); KGGPQVTRGDVFTMP (SEQ ID NO:73); GGPQVTRGDVFTMP(SEQ ID NO:74); GGPQVTRGDVFTMPK (SEQ ID NO:75); GRGDSPK (SEQ ID NO:76); KGGAVTGRGDSPASS(SEQ ID NO:77); GGAVTGRGDSPASS (SEQ ID NO:78); XaaPQVTRGNVFTMP (SEQ ID NO:79); RGDYK (SEQ ID NO:80) or GGVTRGNVFTMP (SEQ ID NO:81) where the peptide sequences may be linear or cyclic, or combinations. In an aspect (56) the apparatus of aspect 38 is provided wherein the apparatus is multi-well plate, a flask, a roller bottle, a multi-layer flask, a bead, a microcarrier, a jar a dish, a beaker, a tube, a cover slip, a bag, a membrane, a hollow fiber, a cup, a spinner bottle, a perfusion chamber, a bioreactor or a fermenter.

In a still further aspect (57), an apparatus for cell culture is provided, comprising: a substrate; a polymer layer on the substrate, the polymer layer comprising a proteolytic enzyme-cleavable amino acid sequence; and a cell adhesive amino acid sequence, wherein the polymer layer is formed from at least two functionalized enzyme-cleavable peptide monomers of the formula: Z-Xaaₙ-Z¹ₙ₁ wherein Z and Z¹ are polymerization moieties and n1 is an integer of 0 or 1, wherein Xaa is each independently an amino acid and n is an integer from 2 to 100, and wherein at least one Xaaₙ is an amino acid sequence that is cleavable by a protease enzyme; and a functionalized cell adhesive peptide monomer of the formula: Z-CAP-Z¹ₙ₁ wherein Z and Z¹ are polymerization moieties and n1 is an integer of 0 or 1, and CAP is a cell adhesive peptide. In an aspect (58), the apparatus of aspect 57 is provided further comprising 3-methacryoyl lysine. In an aspect (59), the apparatus of aspect 57 or 58 is provided further comprising 1-vinyl imidazole. In an aspect (60) the apparatus of any one of aspects 57-59 is provided, wherein the substrate comprises ceramic, glass, plastic, a polymer or co-polymer, any combinations thereof, or a coating of one material on another. In an aspect (61), the apparatus of any one of aspects 57-60 is provided wherein the apparatus is multi-well plate, a flask, a roller bottle, a multi-layer flask, a bead, a microcarrier, a jar a dish, a beaker, a tube, a cover slip, a bag, a membrane, a hollow fiber, a cup, a spinner bottle, a perfusion chamber, a bioreactor or a fermenter. In an aspect (62), the apparatus of any one of aspects 57-61 is provided wherein the polymerization moiety comprises an acrylate, methacrylate, vinyl, acrylamide, methacryalmide, maleimide, epoxide or fumarate group. In an aspect (63), the apparatus of any one of aspects 57-62 is provided wherein Xaaₙ comprises at least one Lys amino acid, and at least one Lys amino acid of Xaaₙ comprises a polymerization moiety at the epsilon nitrogen of the Lys sidechain. In an aspect (64), the apparatus of any one of aspects 57-63 is provided wherein the functionalized enzyme-cleavable peptide monomer comprises a trypsin-cleavable sequence. In an aspect (65), the apparatus of any one of aspects 57-64 is provided wherein one of the at least two monomers is MAA-Lys-Lys-NH₂ (SEQ ID NO:1). In an aspect (66), the apparatus of any one of aspects 57-64 is provided wherein one of the at least two monomers is MAA-Lys-Lys(MAA)-NH₂ (SEQ ID NO:2). IN an aspect (67), the apparatus of any one of aspects 57-66 is provided wherein one of the at least two monomers is MAA-Arg-Arg-NH₂ (SEQ ID NO:5). In an aspect (68) the apparatus of any one of claims 57-64 is provided wherein one of the at least two monomers is MAA-Arg-Arg-Lys(MAA)-NH₂ (SEQ ID NO:3). In an aspect (69) the apparatus of any one of claims 57-66 is provided wherein the enzyme-cleavable amino acid sequence is Lys-Lys (SEQ ID NO: 1) or Arg-Arg (SEQ ID NO: 5). In an aspect (70), apparatus of any one of aspects 57-62 is provided wherein the functionalized enzyme-cleavable peptide monomer comprises a carboxypeptidase-cleavable sequence. In an aspect (71), the apparatus of aspect 70 is provided wherein at least one of the at least two monomers comprises (MAA)-Ala-Tyr-Ala-Phe-OH (SEQ ID NO:8). In an aspect (72), the apparatus of any one of aspects 57-62 is provided, wherein the functionalized enzyme-cleavable peptide monomer comprises a chymotrypsin-cleavable sequence. In an aspect (73) the apparatus of aspect 72 is provided wherein the functionalized enzyme-cleavable peptide monomer comprises (MAA)-Ala-Leu-Phe-Ala-Leu-Arg-NH₂ (SEQ ID NO:9). In an aspect (74), the apparatus of any one of aspects 57-62 is provided wherein the functionalized enzyme-cleavable peptide monomer comprises an elastase-cleavable sequence. In an aspect (75), the apparatus of aspect 74 is provided wherein the functionalized enzyme-cleavable peptide monomer comprises (MAA)-Ala-Ala-Ala-Ala-Leu-Phe-Arg-NH₂ (SEQ ID NO:10), (MAA)-Ala-Ala-Pro-Ala-NH₂ (SEQ ID NO:11), (MAA)-Ala-Ala-Pro-Val-NH₂(SEQ ID NO:12), (MAA)-Ala-Ala-Pro-Met-NH₂ (SEQ ID NO:13) or (MAA)-Arg-Glu-His-Val-Ile-Phe-NH₂ (SEQ ID NO:14). In an aspect (76), the apparatus of any one of aspects 57-62 is provided wherein the functionalized enzyme-cleavable peptide monomer comprises a papain-cleavable sequence. In an aspect (77), the apparatus of aspect 76 is provided wherein the functionalized enzyme-cleavable peptide monomer comprises (MAA)-Ala-Phe-Glu-Leu-Phe-Arg-NH₂, (SEQ ID NO:15). In an aspect (78) the apparatus of any one of aspects 57-62 is provided wherein the functionalized enzyme-cleavable peptide monomer comprises a pepsin-cleavable sequence. In an aspect (79) the apparatus of aspect 78 is provided wherein the functionalized enzyme-cleavable peptide monomer comprises (MAA)-Ala-His-Phe-Phe-Arg-Leu-NH₂ (SEQ ID NO:16). In an aspect (80), the apparatus of any one of aspects 57-62 is provided, wherein the functionalized enzyme-cleavable peptide monomer comprises a plasmin-cleavable sequence. In an aspect (81), the apparatus of aspect 80 is provided wherein the functionalized enzyme-cleavable peptide monomer comprises (MAA)-Lys-Thr-Tyr-Lys-NH₂ (SEQ ID NO:17), (MAA)-Lys-Thr-Phe-Lys-NH₂ (SEQ ID NO:18), (MAA)-Lys-Thr-Trp-Lys-NH₂ (SEQ ID NO:19), (MAA)-Lys-Thr-Ser-Lys-NH₂ (SEQ ID NO:20), (MAA)-Phe-Thr-Tyr-Lys-NH₂ (SEQ ID NO:21), (MAA)-Leu-Thr-Phe-Lys-NH₂ (SEQ ID NO:22), or (MAA)-Leu-Glu-Phe-Lys-NH₂(SEQ ID NO:23). In an aspect (82), the apparatus of any one of aspects 57-62 is provided wherein the functionalized enzyme-cleavable peptide monomer comprises a thrombin-cleavable sequence. In an aspect (83), the apparatus of aspect 82 is provided wherein the functionalized enzyme-cleavable peptide monomer comprises (MAA)-Nleu-Thr-Pro-Arg-NH₂ (SEQ ID NO:24), (MAA)-Val-Thr-Pro-Arg-NH₂ (SEQ ID NO:26), (MAA)-Nleu-Thr-Leu-Arg-NH₂ (SEQ ID NO:27), (MAA)-Leu-Gly-Val-Arg-NH₂ (SEQ ID NO:28) or (MAA)-Gly-Gly-Val-Arg-NH₂ (SEQ ID NO:29). In an aspect (84), the apparatus of any one of aspects 57-62 is provided wherein the functionalized enzyme-cleavable peptide monomer comprises a caspase-cleavable sequence. IN an aspect (85), the apparatus of aspect 84 is provided wherein the functionalized enzyme-cleavable peptide monomer comprises (MAA)-Val-Asp-Val-Ala-Asp-Val-Asp-Val-Ala-Asp-NH₂ (SEQ ID NO:30), (MAA)-Asp-Glu-Val-Asp-Asp-Glu-Val-Asp-NH₂ (SEQ ID NO:31), (MAA)-Ala-Ala-Ala-Asp-Ala-Ala-Asp-NH₂ (SEQ ID NO:32), (MAA)-Leu-Glu-His-Asp-Ala-Ala-Asp-NH₂ (SEQ ID NO:33), (MAA)-Val-Glu-Ile-Asp-Ala-Ala-Asp-NH₂ (SEQ ID NO:34) or (MAA)-Asp-Asp-Asp-NH₂ (SEQ ID NO:35). In an aspect (86), the apparatus of any one of aspects 57-62 is provided wherein the functionalized enzyme-cleavable peptide monomer comprises a collagenase-cleavable sequence. IN an aspect (87), the apparatus of aspect 88 is provided wherein the functionalized enzyme-cleavable peptide monomer comprises (MAA)-Gly-Pro-Gln-Gly-Ile-Ala-Gly-Gln-NH₂ (SEQ ID NO:36), (MAA)-Pro-Ser-Tyr-Phe-Leu-Asn-Ala-Gly-NH₂ (SEQ ID NO:37), (MAA)-Gly-Pro-Leu-Gly-Met-Arg-Gly-Leu-NH₂ (SEQ ID NO:38), (MAA)-Gly-Gly-Pro-Leu-Gly-Pro-Pro-Gly-NH₂ (SEQ ID NO:39). In an aspect (88) the apparatus of any one of aspects 57-62 is provided wherein the functionalized enzyme-cleavable peptide monomer comprises a cathepsin G-cleavable sequence. IN an aspect (89), the apparatus of aspect 88 is provided wherein the functionalized enzyme-cleavable peptide monomer comprises (MAA)-Leu-Leu-Ser-Ala-Leu-Gln-NH₂ (SEQ ID NO:40) or (MAA)-Thr-Leu-Leu-Ser-Ala-Leu-Gln-NH₂ (SEQ ID NO:41). IN an aspect (90) the apparatus of any one of aspects 57-62 is provided, wherein the functionalized enzyme-cleavable peptide monomer comprises a cathepsin D-cleavable sequence. In an aspect (91) the apparatus of aspect 90 is provided wherein the functionalized enzyme-cleavable peptide monomer comprises (MAA)-Glu-Glu-Lys-Pro-Ile-Met-Phe-Phe-Arg-Leu-Leu-Gly-Lys-Lys-NH₂ (SEQ ID NO:42), (MAA)-Glu-Asp-Lys-Pro-Ile-Leu-Phe-Phe-Arg-Leu-Gly-Lys-NH₂ (SEQ ID NO:43) or (MAA)-Glu-Glu-Lys-Pro-Ile-Ser-Phe-Phe-Arg-Leu-Gly-Lys-NH₂ (SEQ ID NO:44). IN an aspect (92) the apparatus of any one of aspects 57-62 is provided wherein the functionalized enzyme-cleavable peptide monomer comprises Cathepsin B cleavable sequence. In an aspect (93), the apparatus of aspect 92 is provided wherein the functionalized enzyme-cleavable peptide monomer comprises (MAA)-Arg-Leu-Arg-Gly-Phe-Glu-NH₂ (SEQ ID NO:45), or (MAA)-Arg-Ile-Ile-Glu-Gly-Ile-Glu-NH₂ (SEQ ID NO:46). In an aspect (93), the apparatus of any one of aspects 57-92 is provided wherein the cell adhesive peptide is selected from the group consisting of KGGGQKCIVQTTSWSQCSKS (SEQ ID NO:47); GGGQKCIVQTTSWSQCSKS(SEQ ID NO:48); KYGLALERKDHSG (SEQ ID NO:49); YGLALERKDHSG (SEQ ID NO:50); KGGSINNNRHSIYITRFGNMGS (SEQ ID NO:51); GGSINNNRHSIYITRFGNMGS (SEQ ID NO:52); KGGTWYKIAFQRNRK (SEQ ID NO:53); GGTWYKIAFQRNRK (SEQ ID NO:54); KGGTSIKIRGTYSER (SEQ ID NO:55); GGTSIKIRGTYSER (SEQ ID NO:56); KYGTDIRVTLNRLNTF (SEQ ID NO:57); YGTDIRVTLNRLNTF (SEQ ID NO:58); KYGSETTVKYIFRLHE (SEQ ID NO:59); YGSETTVKYIFRLHE(SEQ ID NO:60); KYGKAFDITYVRLKF (SEQ ID NO:61); YGKAFDITYVRLKF(SEQ ID NO:62); KYGAASIKVAVSADR (SEQ ID NO:63); YGAASIKVAVSADR(SEQ ID NO:64); KGGNGEPRGDTYRAY(SEQ ID NO:65); GGNGEPRGDTYRAY (SEQ ID NO:66) CGGNGEPRGDTRAY (SEQ ID NO:67); GGNGEPRGDTRAY (SEQ ID NO:68); KYGRKRLQVQLSIRT (SEQ ID NO:69); YGRKRLQVQLSIRT(SEQ ID NO:70); KGGRNIAEIIKDI (SEQ ID NO:71); GGRNIAEIIKDI (SEQ ID NO:72); KGGPQVTRGDVFTMP (SEQ ID NO:73); GGPQVTRGDVFTMP(SEQ ID NO:74); GGPQVTRGDVFTMPK (SEQ ID NO:75); GRGDSPK (SEQ ID NO:76); KGGAVTGRGDSPASS(SEQ ID NO:77); GGAVTGRGDSPASS (SEQ ID NO:78); XaaPQVTRGNVFTMP (SEQ ID NO:79); RGDYK (SEQ ID NO:80), GGVTRGNVFTMP(SEQ ID NO:81), where the peptide sequences may be linear or cyclic, or combinations.

As alternative descriptions of the aspects mentioned above, embodiment 1 is an apparatus for cell culture comprising:
a substrate;
a polymer layer on the substrate, the polymer layer comprising a proteolytic enzyme-cleavable amino acid sequence; and a cell adhesive amino acid sequence,
wherein the polymer layer is formed from at least two functionalized enzyme-cleavable peptide monomers of the formula:
Z-Xaaₙ-Z¹ₙ₁
wherein Z and Z¹ are polymerization moieties and n1 is an integer of 0 or 1,
wherein Xaa is each independently an amino acid and n is an integer from 2 to 100, and wherein at least one Xaaₙ is an amino acid sequence that is cleavable by a protease enzyme;
and a functionalized cell adhesive peptide monomer of the formula:
Z-CAP-Z¹ₙ₁
wherein Z and Z¹ are polymerization moieties and n1 is an integer of 0 or 1, and CAP is a cell adhesive peptide.

Embodiment 2 can be the apparatus of embodiment 1 further comprising 3-methacryoyl lysine.

Embodiment 3 can be the apparatus of embodiments 1 or 2 further comprising 1-vinyl imidazole.

Embodiment 4 can be the apparatus of any one of embodiments 1-3 wherein the substrate comprises ceramic, glass, plastic, a polymer or co-polymer, any combinations thereof, or a coating of one material on another.

Embodiment 5 can be the apparatus of any one of embodiments 1-4 wherein the apparatus is multi-well plate, a flask, a roller bottle, a multi-layer flask, a bead, a microcarrier, a jar a dish, a beaker, a tube, a cover slip, a bag, a membrane, a hollow fiber, a cup, a spinner bottle, a perfusion chamber, a bioreactor or a fermenter.

Embodiment 6 can be the apparatus of any one of embodiments 1-5 wherein the polymerization moiety comprises an acrylate, methacrylate, vinyl, acrylamide, methacryalmide, maleimide, epoxide or fumarate group.

Embodiment 7 can be the apparatus of any one of embodiments 1-6 wherein Xaaₙ comprises at least one Lys amino acid, and at least one Lys amino acid of Xaaₙ comprises a polymerization moiety at the epsilon nitrogen of the Lys sidechain.

Embodiment 8 can be the apparatus of any one of embodiments 1-7 wherein the functionalized enzyme-cleavable peptide monomer comprises a trypsin-cleavable sequence.

Embodiment 9 can be the apparatus of any one of embodiments 1-8 wherein one of the at least two monomers is MAA-Lys-Lys-NH₂ (SEQ ID NO:1).

Embodiment 10 can be the apparatus of any one of embodiments 1-8 wherein one of the at least two monomers is MAA-Lys-Lys(MAA)-NH₂ (SEQ ID NO:2).

Embodiments 11 can be the apparatus of any one of embodiments 1-8 wherein one of the at least two monomers is MAA-Arg-Arg-NH₂ (SEQ ID NO:5).

Embodiment 12 can be the apparatus of any one of embodiments 1-8 wherein one of the at least two monomers is MAA-Arg-Arg-Lys(MAA)-NH₂ (SEQ ID NO:3).

Embodiment 13 can be the apparatus of any one of embodiments 1-8 wherein the enzyme-cleavable amino acid sequence is Lys-Lys (SEQ ID NO: 1) or Arg-Arg (SEQ ID NO: 5).

Embodiment 14 can be the apparatus of any one of embodiments 1-6 wherein the functionalized enzyme-cleavable peptide monomer comprises a carboxypeptidase-cleavable sequence.

Embodiment 15 can be the apparatus of embodiment 14 wherein at least one of the at least two monomers comprises (MAA)-Ala-Tyr-Ala-Phe-OH (SEQ ID NO:8).

Embodiment 16 can be the apparatus of any one of embodiments 1-6 wherein the functionalized enzyme-cleavable peptide monomer comprises a chymotrypsin-cleavable sequence.

Embodiment 17 can be the apparatus of embodiment 16 wherein the functionalized enzyme-cleavable peptide monomer comprises (MAA)-Ala-Leu-Phe-Ala-Leu-Arg-NH₂ (SEQ ID NO:9).

Embodiment 18 can be the apparatus of any one of embodiments 1-6 wherein the functionalized enzyme-cleavable peptide monomer comprises an elastase-cleavable sequence.

Embodiment 19 can be the apparatus of embodiment 18 wherein the functionalized enzyme-cleavable peptide monomer comprises (MAA)-Ala-Ala-Ala-Ala-Leu-Phe-Arg-NH₂ (SEQ ID NO:10), (MAA)-Ala-Ala-Pro-Ala-NH₂ (SEQ ID NO:11), (MAA)-Ala-Ala-Pro-Val-NH₂(SEQ ID NO:12), (MAA)-Ala-Ala-Pro-Met-NH₂ (SEQ ID NO:13) or (MAA)-Arg-Glu-His-Val-Ile-Phe-NH₂ (SEQ ID NO:14).

Embodiment 20 can be the apparatus of any one of embodiments 1-6 wherein the functionalized enzyme-cleavable peptide monomer comprises a papain-cleavable sequence.

Embodiment 21 can be the apparatus of embodiment 20 wherein the functionalized enzyme-cleavable peptide monomer comprises (MAA)-Ala-Phe-Glu-Leu-Phe-Arg-NH₂, (SEQ ID NO:15).

Embodiment 22 can be the apparatus of any one of embodiments 1-6 wherein the functionalized enzyme-cleavable peptide monomer comprises a pepsin-cleavable sequence

Embodiment 23 can be the apparatus of embodiment 22 wherein the functionalized enzyme-cleavable peptide monomer comprises (MAA)-Ala-His-Phe-Phe-Arg-Leu-NH₂ (SEQ ID NO:16).

Embodiment 24 can be the apparatus of any one of embodiments 1-6 wherein the functionalized enzyme-cleavable peptide monomer comprises a plasmin-cleavable sequence.

Embodiment 25 can be the apparatus of embodiment 24 wherein the functionalized enzyme-cleavable peptide monomer comprises (MAA)-Lys-Thr-Tyr-Lys-NH₂ (SEQ ID NO:17), (MAA)-Lys-Thr-Phe-Lys-NH₂ (SEQ ID NO:18), (MAA)-Lys-Thr-Trp-Lys-NH₂ (SEQ ID NO:19), (MAA)-Lys-Thr-Ser-Lys-NH₂ (SEQ ID NO:20), (MAA)-Phe-Thr-Tyr-Lys-NH₂ (SEQ ID NO:21), (MAA)-Leu-Thr-Phe-Lys-NH₂ (SEQ ID NO:22), or (MAA)-Leu-Glu-Phe-Lys-NH₂ (SEQ ID NO:23).

Embodiment 26 can be the apparatus of any one of embodiments 1-6 wherein the functionalized enzyme-cleavable peptide monomer comprises a thrombin-cleavable sequence.

Embodiment 27 can be the apparatus of embodiment 26 wherein the functionalized enzyme-cleavable peptide monomer comprises (MAA)-Nleu-Thr-Pro-Arg-NH₂ (SEQ ID NO:24), (MAA)-Val-Thr-Pro-Arg-NH₂ (SEQ ID NO:26), (MAA)-Nleu-Thr-Leu-Arg-NH₂ (SEQ ID NO:27), (MAA)-Leu-Gly-Val-Arg-NH₂ (SEQ ID NO:28) or (MAA)-Gly-Gly-Val-Arg-NH₂ (SEQ ID NO:29).

Embodiment 28 can be the apparatus of any one of embodiments 1-6 wherein the functionalized enzyme-cleavable peptide monomer comprises a caspase-cleavable sequence.

Embodiment 29 can be the apparatus of embodiment 28 wherein the functionalized enzyme-cleavable peptide monomer comprises (MAA)-Val-Asp-Val-Ala-Asp-Val-Asp-Val-Ala-Asp-NH₂ (SEQ ID NO:30), (MAA)-Asp-Glu-Val-Asp-Asp-Glu-Val-Asp-NH₂ (SEQ ID NO:31), (MAA)-Ala-Ala-Ala-Asp-Ala-Ala-Asp-NH₂ (SEQ ID NO:32), (MAA)-Leu-Glu-His-Asp-Ala-Ala-Asp-NH₂ (SEQ ID NO:33), (MAA)-Val-Glu-Ile-Asp-Ala-Ala-Asp-NH₂ (SEQ ID NO:34) or (MAA)-Asp-Asp-Asp-NH₂ (SEQ ID NO:35).

Embodiment 30 can be the apparatus of any one of embodiments 1-6 wherein the functionalized enzyme-cleavable peptide monomer comprises a collagenase-cleavable sequence.

Embodiment 31 can be the apparatus of embodiment 30 wherein the functionalized enzyme-cleavable peptide monomer comprises (MAA)-Gly-Pro-Gln-Gly-Ile-Ala-Gly-Gln-NH₂ (SEQ ID NO:36), (MAA)-Pro-Ser-Tyr-Phe-Leu-Asn-Ala-Gly-NH₂ (SEQ ID NO:37), (MAA)-Gly-Pro-Leu-Gly-Met-Arg-Gly-Leu-NH₂ (SEQ ID NO:38), (MAA)-Gly-Gly-Pro-Leu-Gly-Pro-Pro-Gly-NH₂(SEQ ID NO:39).

Embodiment 32 can be the apparatus of any one of embodiments 1-6 wherein the functionalized enzyme-cleavable peptide monomer comprises a cathepsin G-cleavable sequence.

Embodiment 33 The apparatus of embodiment 32 wherein the functionalized enzyme-cleavable peptide monomer comprises (MAA)-Leu-Leu-Ser-Ala-Leu-Gln-NH₂ (SEQ ID NO:40) or (MAA)-Thr-Leu-Leu-Ser-Ala-Leu-Gln-NH₂ (SEQ ID NO:41).

Embodiment 34 can be the apparatus of any one of embodiments 1-6 wherein the functionalized enzyme-cleavable peptide monomer comprises a cathepsin D-cleavable sequence.

Embodiment 35 can be the apparatus of embodiment 34 wherein the functionalized enzyme-cleavable peptide monomer comprises (MAA)-Glu-Glu-Lys-Pro-Ile-Met-Phe-Phe-Arg-Leu-Leu-Gly-Lys-Lys-NH₂ (SEQ ID NO:42), (MAA)-Glu-Asp-Lys-Pro-Ile-Leu-Phe-Phe-Arg-Leu-Gly-Lys-NH₂ (SEQ ID NO:43) or (MAA)-Glu-Glu-Lys-Pro-Ile-Ser-Phe-Phe-Arg-Leu-Gly-Lys-NH₂ (SEQ ID NO:44).

Embodiment 36 can be the apparatus of any one of embodiments 1-6 wherein the functionalized enzyme-cleavable peptide monomer comprises Cathepsin B cleavable sequence.

Embodiment 37 can be the apparatus of embodiment 36 wherein the functionalized enzyme-cleavable peptide monomer comprises (MAA)-Arg-Leu-Arg-Gly-Phe-Glu-NH₂ (SEQ ID NO:45), or (MAA)-Arg-Ile-Ile-Glu-Gly-Ile-Glu-NH₂ (SEQ ID NO:46).

Embodiment 38 can be the apparatus of any one of embodiments 1-37 wherein the cell adhesive peptide is selected from the group consisting of KGGGQKCIVQTTSWSQCSKS (SEQ ID NO:47); GGGQKCIVQTTSWSQCSKS(SEQ ID NO:48); KYGLALERKDHSG (SEQ ID NO:49); YGLALERKDHSG (SEQ ID NO:50); KGGSINNNRHSIYITRFGNMGS (SEQ ID NO:51); GGSINNNRHSIYITRFGNMGS (SEQ ID NO:52); KGGTWYKIAFQRNRK (SEQ ID NO:53); GGTWYKIAFQRNRK (SEQ ID NO:54); KGGTSIKIRGTYSER (SEQ ID NO:55); GGTSIKIRGTYSER (SEQ ID NO:56); KYGTDIRVTLNRLNTF (SEQ ID NO:57); YGTDIRVTLNRLNTF (SEQ ID NO:58); KYGSETTVKYIFRLHE (SEQ ID NO:59); YGSETTVKYIFRLHE(SEQ ID NO:60); KYGKAFDITYVRLKF (SEQ ID NO:61); YGKAFDITYVRLKF(SEQ ID NO:62); KYGAASIKVAVSADR (SEQ ID NO:63); YGAASIKVAVSADR(SEQ ID NO:64); KGGNGEPRGDTYRAY(SEQ ID NO:65); GGNGEPRGDTYRAY (SEQ ID NO:66) CGGNGEPRGDTRAY (SEQ ID NO:67); GGNGEPRGDTRAY (SEQ ID NO:68); KYGRKRLQVQLSIRT (SEQ ID NO:69); YGRKRLQVQLSIRT(SEQ ID NO:70); KGGRNIAEIIKDI (SEQ ID NO:71); GGRNIAEIIKDI (SEQ ID NO:72); KGGPQVTRGDVFTMP (SEQ ID NO:73); GGPQVTRGDVFTMP(SEQ ID NO:74); GGPQVTRGDVFTMPK (SEQ ID NO:75); GRGDSPK (SEQ ID NO:76); KGGAVTGRGDSPASS(SEQ ID NO:77); GGAVTGRGDSPASS (SEQ ID NO:78); XaaPQVTRGNVFTMP (SEQ ID NO:79); RGDYK (SEQ ID NO:80), GGVTRGNVFTMP(SEQ ID NO:81), where the peptide sequences may be linear or cyclic, or combinations.

In the following, non-limiting examples are presented, which describe various embodiments of the articles and methods discussed above.

### EXAMPLES

Materials: Photoinitiators Irgacure-819 (Phosphine oxide, phenyl bis(2,4,6-trimethyl benzoyl) and Darocur 1173 (2-hydroxy-2-methyl-1-phenyl-1-propanone) used in the free radical polymerization of the acrylate hydrogel formulations were obtained from Ciba Specialty Chemicals (Newport Del.) and used without any further purification. Vinyl-imidazole and 3-Methacryoyl Lysine were purchased from Polyscience Corporation (Niles, Il) and used as received. Ethanol 200 proof was available in house and used as a solvent to prepare formulations.

General Process for the Synthesis of Functionalized Peptides:

Preparation of (MAA)Lys-Lys-NH₂ (SEQ ID NO:1): This functionalized cationic peptide monomer was synthesized on 0.5 mmol Rink Amide resin via standard Fmoc chemistry. The side-chain protecting group, Boc, was used for the amino acid Lys. Fmoc N-protected amino acids were purchased from GL Biochem. Methacrylic acid (MAA) and the coupling and cleavage reagents were purchased from Aldrich. Solvents were purchased from Fisher Scientific. The peptide chain was assembled on resin beginning at the C-terminus by repetitive removal of the Fmoc N-protecting group, followed by subsequent coupling of each N-protected amino acid. HBTU and HOBt were used as coupling reagents and NMM was used as base. A 20% solution of piperidine in DMF was used as the de-Fmoc-reagent. After completion of the last coupling, the peptide resin was treated with TFA cleavage cocktail for 3 hours to cleave the peptide from the resin and remove the side-chain protecting groups. The resulting crude peptide was precipitated from cold ether and dried under vacuum. Yield 500 mg, purity >80%. A total of 500 mg of crude peptide was purified by a 2-inch Waters C18 column with TFA buffer (0.1% TFA in water). Resulting fractions with purity of >90% were lyophilized to dryness. A total of 200 mg of final peptide with a purity of 98.0% was obtained. The product was provided by American Peptide and was used without further purification.

Preparation of (MAA)-Arg-Arg-NH₂ (SEQ ID NO:5): This peptide was synthesized on 0.5 mmol Rink Amide resin via standard Fmoc chemistry. The side-chain protecting group, Pbf, was used for the amino acid Arg. Fmoc N-protected amino acids were purchased from GL Biochem. Methacrylic acid (MAA) and the reagents for coupling and cleavage were purchased from Aldrich. Solvents were purchased from Fisher Scientific. The peptide chain was assembled on resin, beginning at the C-terminal, by repetitive removal of the Fmoc N-protecting groups followed by subsequent coupling of each N-protected amino acid. HBTU and HOBt were used as coupling reagent and NMM was used as base. A 20% solution of piperidine in DMF was used as the de-Fmoc-reagent. After completion of the last coupling, the peptide resin was treated with TFA cleavage cocktail for 3 hours to cleave the peptide from the resin and remove the side-chain protecting groups. The resulting crude peptide was precipitated from cold ether and dried under vacuum. Yield 800 mg, purity >70%. Approximately 800 mg of crude peptide was purified by a 2-inch C18 column with TFA buffer (0.1% TFA in water) using a linear gradient of 0-30% acetonitrile in 60 minutes. The pooled fractions with purity of >90% were lyophilized to dryness. A total of 279 mg of final peptide with a purity of 95.3% was obtained. The product was provided by American Peptide and was used without further purification.

Preparation of (MAA)-Lys-Lys-Lys(MAA)-NH₂ (SEQ ID NO:4): This peptide was synthesized on 1.0 mmol Rink Amide resin via standard Fmoc chemistry. The side-chain protecting groups used for amino acids were Mtt for C-terminal Lys and Boc for all other Lys residues. Fmoc N-protected amino acids were purchased from GL Biochem. Methacrylic acid (MAA) and the reagents for coupling and cleavage were purchased from Aldrich. Solvents were purchased from Fisher Scientific. The peptide chain was assembled on resin beginning at the C-terminus by repetitive removal of the Fmoc N-protecting groups, followed by subsequent coupling of each N-protected amino acid. HBTU and HOBt were used as coupling reagents and NMM was used as base. A 20% solution of piperidine in DMF was used as the de-Fmoc-reagent. After removal of the Fmoc protecting group at the N-terminal Lys, the Mtt side-chain protecting group was removed by 1% TFA in DCM. The MAA was coupled on the amino group of the N-terminal Lys and on the side-chain of the C-terminal Lys. Peptide resin was treated with TFA cleavage cocktail for 3 hours to cleave the peptide from the resin and remove the Boc Lys side-chain protecting groups. The resulting crude peptide was precipitated from cold ether and dried under vacuum. Yield 1300 mg, purity >20%. The product was provided by American Peptide and was used without further purification. Other functionalized cationic peptide monomers were prepared in a similar fashion.

General Procedure for the preparation of functionalized enzyme-cleavable peptide polymer formulations in 96 well plates: To make PLYS-1, into each well of a 96 well plate, 130 μg of MAA-Lys-Lys-NH₂ (SEQ ID NO: 1), 26 μg of MAA-Lys-Lys-Lys(MAA)-NH₂ (SEQ ID NO: 4), 0.55 μg of 3-methacryoyl lysine, 0.28 μg of 1-vinyl imidazole and 1.05 μg of VN-methacrylate (0.25 mM) were added. To make PARG-1, 50 μg of MAA-Arg-Arg-NH₂ (SEQ ID NO: 5), 5.2 μg of MAA-Lys-Lys(MAA)-NH₂ (SEQ ID NO: 2), 0.10 μg of 3-methacryoyl lysine, 0.10 μg of 1-vinyl imidazole and 1.05 μg VN-methacrylate were added to each well.

General procedure for preparing stock solutions of monomers: Monomers were added to a 20 ml glass vial in proportions shown in Tables 3 and 4. A 100 ml solution of ethanol containing 1% 1-819 and 10% D-1173 was mixed as a stock solution and used to prepare formulations according to concentrations represented in tables 3-4. Other functionalized cationic peptide polymers were similarly prepared.

General procedure for UV curing functionalized cationic peptide polymer formulations: A “Xenon Model RC-801 high intensity pulsed Ultraviolet (UV) light curing system” from INPRO Technologies, Inc. was used in curing. The plates were constantly being purged with nitrogen in order to create an inert environment (for the coatings) during curing. The cure time was set (i.e. 60 sec. in this study).

Procedure for culturing cells: Bone marrow derived human mesenchymal stem cells were purchased from Millipore Corporation (Billerica, Mass.). These cells were gently thawed at 37° C. water bath until almost completely thawed. The cells were added to 0.1% gelatin coated T175 flask containing approximately 25 ml of hMSC chemically defined Mesencult0XF media from Stem Cell Technologies. The flask was transferred to humidified incubator set to 37° C. and 5% CO2 and 95% humidity. Fresh medium was added every alternate day and spent media was removed and discarded. The hMSC cultures were passaged once 80% confluence was reached by visual inspection under a microscope. For passaging cells were harvested using 0.05% trypsin with 1 mM EDTA as a dissociation agent. Trypsin was removed by centrifugating the cell suspension in Trypsin at 210 g for 5 minutes. The cell pellet obtained was immediately added with fresh chemically defined media brought to 37° C.

General procedure for assaying cell release: At 80% confluency, hMSC were first washed with DPBS without Calcium and magnesium. Established cell cultures were exposed to lytic agents such as 0.25% trypsin with 1 mM EDTA (available from Millipore, Billerica, Mass.) or commercially available such as MesenCult®-ACF Dissociation Kit (Catalog #05426), available from Stemcell Technologies (Vancouver, BC)

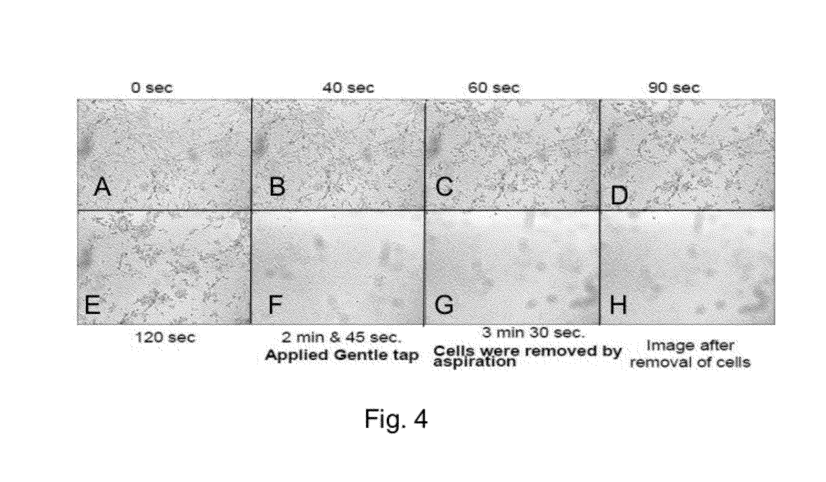

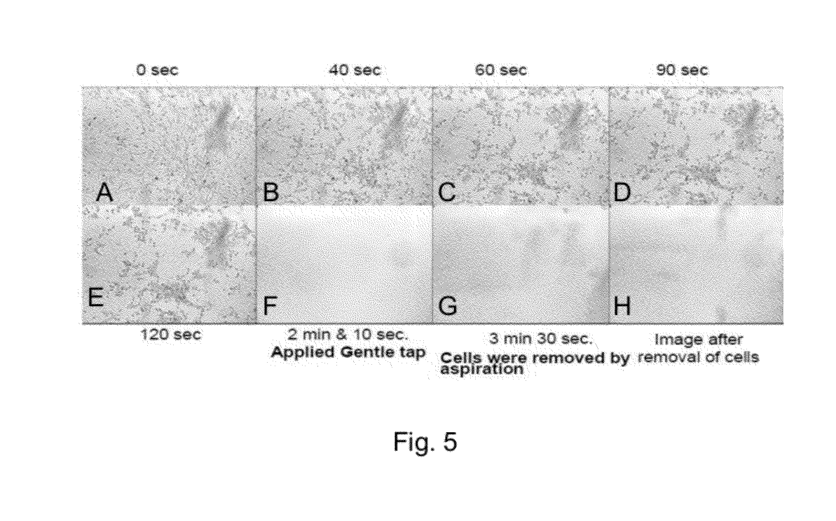

FIG. 4A-H are photographs showing morphology of human mesenchymal stem cells, cultured on the PARG-1-VN (as shown in Table 3) embodiment of the cell culture surface disclosed herein, after treatment with a proteolytic enzyme, at zero seconds (A), forty seconds (B), sixty seconds (C), ninety seconds (D), one hundred twenty seconds (E), 2 minutes and forty five seconds (F), three minutes and thirty seconds (G) and after removal of cells (H). Human mesenchymal stem cells, shown in FIG. 3A exhibit normal morphology prior to treatment with proteolytic dissociation solution from Stem cell Technologies. Similar results were shown using 25% trypsin with 1 mM EDTA. The cells are released from the surface within three minutes. FIG. 5A-H are photographs showing morphology of human mesenchymal stem cells, cultured on a PARG-1-VN embodiment of the cell culture surface disclosed herein, after treatment with Trypsin and 1 mM EDTA, at zero seconds (A), forty seconds (B), sixty seconds (C), ninety seconds (D), one hundred twenty seconds (E), 2 minutes and forty five seconds (F), three minutes and thirty seconds (G) and after removal of cells (H).

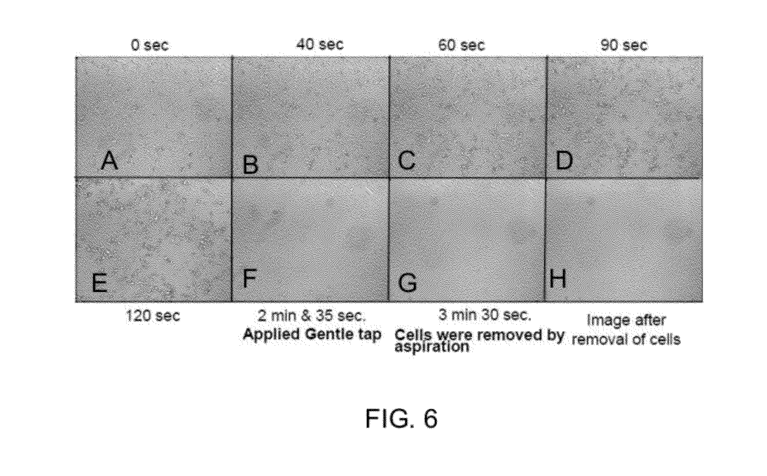

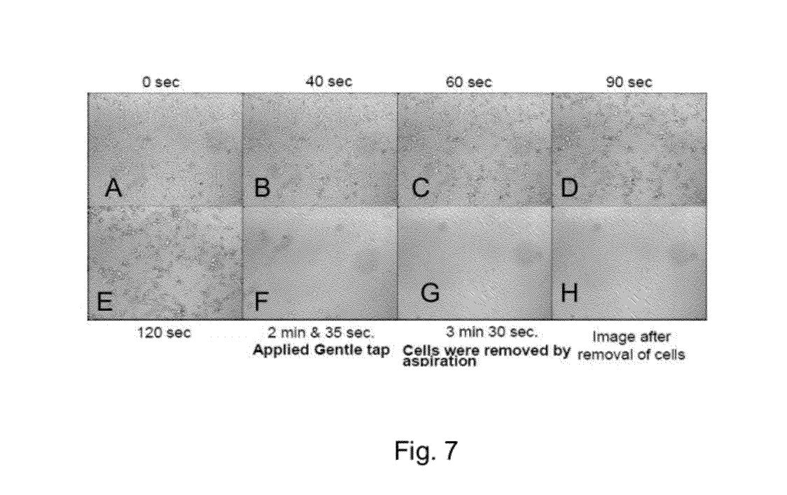

FIG. 6 A-H are photographs showing morphology of human mesenchymal stem cells, cultured on the PLYS-1-VN embodiment of the cell culture surface disclosed herein (as shown in Table 4), after treatment with a proteolytic enzyme, at zero seconds (A), forty seconds (B), sixty seconds (C), ninety seconds (D), one hundred twenty seconds (E), 2 minutes and thirty five seconds (F), three minutes and thirty seconds (G) and after removal of cells (H). Human mesenchymal stem cells, shown in FIG. 4A exhibit normal morphology prior to treatment with proteolytic dissociation solution from Stem cell Technologies. Similar results were shown using 25% trypsin with 1 mM EDTA. The cells are released from the surface within three minutes. FIG. 7 A-H are photographs showing morphology of human mesenchymal stem cells, cultured on PLYS-1-VN embodiment of the cell culture surface disclosed herein, after treatment with Trypsin and 1 mM EDTA, at zero seconds (A), forty seconds (B), sixty seconds (C), ninety seconds (D), one hundred twenty seconds (E), 2 minutes and thirty five seconds (F), three minutes and thirty seconds (G) and after removal of cells (H).

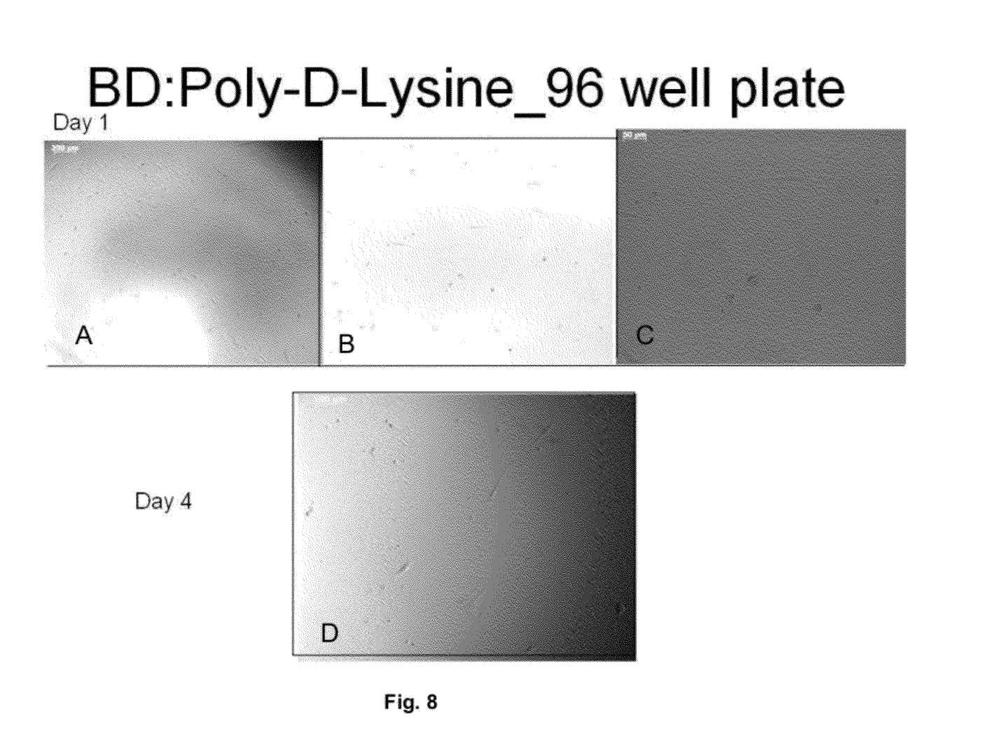

FIG. 8A-D are photographs showing morphology of human mesenchymal stem cells, cultured on comparative example of a cell culture surface (Poly-D-Lysine, available from BD biosciences, Franklin Lakes, JN) after one day (A-C) and after four days (D) of culture. FIG. 5 illustrates that human mesenchymal stem cells did not adhere to the Poly-D-Lysine substrate.

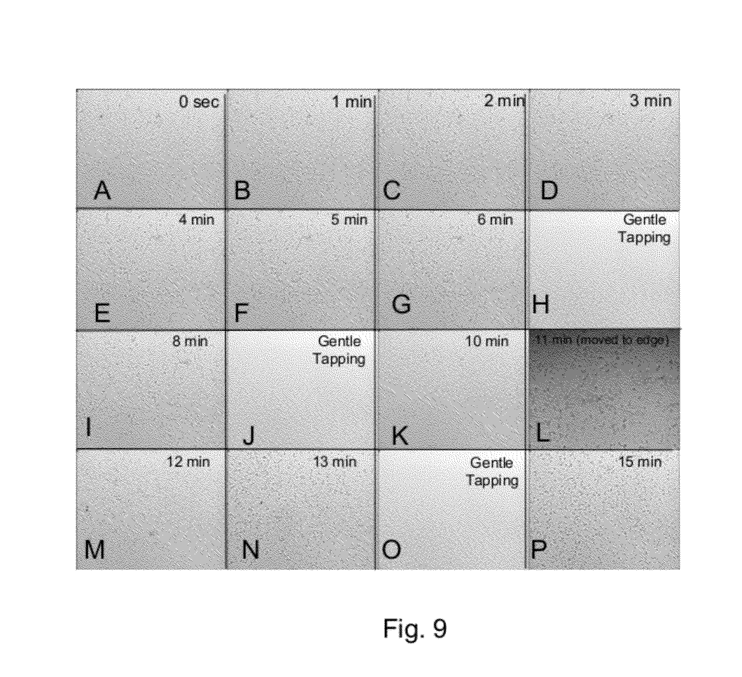

FIG. 9 A-P are photographs showing morphology of human mesenchymal stem cells, cultured on comparative example of a cell culture surface (Synthemax™ available from Corning Incorporated, Corning, N.Y.) after treatment with a proteolytic enzyme, at time zero seconds (A); one minute (B); two minutes (C); three minutes (D); four minutes (E); five minutes (F); six minutes (G); after gentle tapping after six minutes (H); after eight minutes (I); after gentle tapping after eight minutes (K); after eleven minutes (L), after 12 minutes (M); after thirteen minutes (N); after gentle tapping after thirteen minutes (O); and after fifteen minutes (P). While the human embryonic mesenchymal cells adhered to the surface, they did not release, even after fifteen minutes of exposure.

Thus, embodiments of ENZYME CLEAVABLE CELL RELEASE POLYMERIC SURFACE are disclosed. One skilled in the art will appreciate that the arrays, compositions, kits articles and methods described herein can be practiced with embodiments other than those disclosed. The disclosed embodiments are presented for purposes of illustration and not limitation.

What is claimed is:

1. An apparatus for cell culture comprising:
a substrate;
a polymer layer on the substrate, the polymer layer comprising a proteolytic enzyme-cleavable amino acid sequence; and a cell adhesive amino acid sequence,
wherein the polymer layer is formed from at least two functionalized enzyme-cleavable peptide monomers of the formula:
Z-Xaaₙ-Z¹ₙ₁
wherein Z and Z¹ are polymerization moieties and n1 is an integer of 0 or 1, wherein Xaa is each independently an amino acid and n is an integer from 2 to 100, and wherein at least one Xaaₙ is an amino acid sequence that is cleavable by a protease enzyme;
and a functionalized cell adhesive peptide monomer of the formula:
Z-CAP-Z¹ₙ₁
wherein Z and Z¹ are polymerization moieties and n1 is an integer of 0 or 1, and CAP is a cell adhesive peptide.

2. The apparatus of claim 1 further comprising 3-methacryoyl lysine.

3. The apparatus of claim 1 or 2 further comprising 1-vinyl imidazole.

4. The apparatus of any one of claims 1-3 claim 1 wherein the substrate comprises ceramic, glass, plastic, a polymer or co-polymer, any combinations thereof, or a coating of one material on another.

5. The apparatus of any one of claims 1-4 wherein the apparatus is multi-well plate, a flask, a roller bottle, a multi-layer flask, a bead, a microcarrier, a jar a dish, a beaker, a tube, a cover slip, a bag, a membrane, a hollow fiber, a cup, a spinner bottle, a perfusion chamber, a bioreactor or a fermenter.

6. The apparatus of any one of claims 1-5 wherein the polymerization moiety comprises an acrylate, methacrylate, vinyl, acrylamide, methacryalmide, maleimide, epoxide or fumarate group.

7. The apparatus of any one of claims 1-6 wherein Xaaₙ comprises at least one Lys amino acid, and at least one Lys amino acid of Xaaₙ comprises a polymerization moiety at the epsilon nitrogen of the Lys sidechain.

8. The apparatus of any one of claims 1-7 wherein the functionalized enzyme-cleavable peptide monomer comprises a trypsin-cleavable sequence.

9. The apparatus of any one of claims 1-8 wherein one of the at least two monomers is MAA-Lys-Lys-NH₂ (SEQ ID NO:1).

10. The apparatus of any one of claims 1-8 wherein one of the at least two monomers is MAA-Lys-Lys(MAA)-NH₂ (SEQ ID NO:2).

11. The apparatus of any one of claims 1-8 wherein one of the at least two monomers is MAA-Arg-Arg-NH₂ (SEQ ID NO:5).

12. The apparatus of any one of claims 1-8 wherein one of the at least two monomers is MAA-Arg-Arg-Lys(MAA)-NH₂ (SEQ ID NO:3).

13. The apparatus of any one of claims 1-8 wherein the enzyme-cleavable amino acid sequence is Lys-Lys (SEQ ID NO: 1) or Arg-Arg (SEQ ID NO: 5).

14. The apparatus of any one of claims 1-6 wherein the functionalized enzyme-cleavable peptide monomer comprises a carboxypeptidase-cleavable sequence.

15. The apparatus of claim 14 wherein at least one of the at least two monomers comprises (MAA)-Ala-Tyr-Ala-Phe-OH (SEQ ID NO:8).

16. The apparatus of any one of claims 1-6 wherein the functionalized enzyme-cleavable peptide monomer comprises a chymotrypsin-cleavable sequence.

17. The apparatus of claim 16 wherein the functionalized enzyme-cleavable peptide monomer comprises (MAA)-Ala-Leu-Phe-Ala-Leu-Arg-NH₂ (SEQ ID NO:9).

18. The apparatus of any one of claims 1-6 wherein the functionalized enzyme-cleavable peptide monomer comprises an elastase-cleavable sequence.

19. The apparatus of claim 18 wherein the functionalized enzyme-cleavable peptide monomer comprises (MAA)-Ala-Ala-Ala-Ala-Leu-Phe-Arg-NH₂ (SEQ ID NO:10), (MAA)-Ala-Ala-Pro-Ala-NH₂ (SEQ ID NO:11), (MAA)-Ala-Ala-Pro-Val-NH₂(SEQ ID NO:12), (MAA)-Ala-Ala-Pro-Met-NH₂ (SEQ ID NO:13) or (MAA)-Arg-Glu-His-Val-Ile-Phe-NH₂ (SEQ ID NO:14).

20. The apparatus of any one of claims 1-6 wherein the functionalized enzyme-cleavable peptide monomer comprises a papain-cleavable sequence.

21. The apparatus of claim 20 wherein the functionalized enzyme-cleavable peptide monomer comprises (MAA)-Ala-Phe-Glu-Leu-Phe-Arg-NH₂, (SEQ ID NO:15).

22. The apparatus of any one of claims 1-6 wherein the functionalized enzyme-cleavable peptide monomer comprises a pepsin-cleavable sequence

23. The apparatus of claim 22 wherein the functionalized enzyme-cleavable peptide monomer comprises (MAA)-Ala-His-Phe-Phe-Arg-Leu-NH₂ (SEQ ID NO:16).

24. The apparatus of any one of claims 1-6 wherein the functionalized enzyme-cleavable peptide monomer comprises a plasmin-cleavable sequence.

25. The apparatus of claim 24 wherein the functionalized enzyme-cleavable peptide monomer comprises (MAA)-Lys-Thr-Tyr-Lys-NH₂ (SEQ ID NO:17), (MAA)-Lys-Thr-Phe-Lys-NH₂ (SEQ ID NO:18), (MAA)-Lys-Thr-Trp-Lys-NH₂ (SEQ ID NO:19), (MAA)-Lys-Thr-Ser-Lys-NH₂ (SEQ ID NO:20), (MAA)-Phe-Thr-Tyr-Lys-NH₂ (SEQ ID NO:21), (MAA)-Leu-Thr-Phe-Lys-NH₂ (SEQ ID NO:22), or (MAA)-Leu-Glu-Phe-Lys-NH₂ (SEQ ID NO:23).

26. The apparatus of any one of claims 1-6 wherein the functionalized enzyme-cleavable peptide monomer comprises a thrombin-cleavable sequence.

27. The apparatus of claim 26 wherein the functionalized enzyme-cleavable peptide monomer comprises (MAA)-Nleu-Thr-Pro-Arg-NH₂ (SEQ ID NO:24), (MAA)-Val-Thr-Pro-Arg-NH₂ (SEQ ID NO:26), (MAA)-Nleu-Thr-Leu-Arg-NH₂ (SEQ ID NO:27), (MAA)-Leu-Gly-Val-Arg-NH₂ (SEQ ID NO:28) or (MAA)-Gly-Gly-Val-Arg-NH₂ (SEQ ID NO:29).

28. The apparatus of any one of claims 1-6 wherein the functionalized enzyme-cleavable peptide monomer comprises a caspase-cleavable sequence.

29. The apparatus of claim 28 wherein the functionalized enzyme-cleavable peptide monomer comprises (MAA)-Val-Asp-Val-Ala-Asp-Val-Asp-Val-Ala-Asp-NH₂ (SEQ ID NO:30), (MAA)-Asp-Glu-Val-Asp-Asp-Glu-Val-Asp-NH₂ (SEQ ID NO:31), (MAA)-Ala-Ala-Ala-Asp-Ala-Ala-Asp-NH₂ (SEQ ID NO:32), (MAA)-Leu-Glu-His-Asp-Ala-Ala-Asp-NH₂ (SEQ ID NO:33), (MAA)-Val-Glu-Ile-Asp-Ala-Ala-Asp-NH₂ (SEQ ID NO:34) or (MAA)-Asp-Asp-Asp-NH₂ (SEQ ID NO:35).

30. The apparatus of any one of claims 1-6 wherein the functionalized enzyme-cleavable peptide monomer comprises a collagenase-cleavable sequence.

31. The apparatus of claim 30 wherein the functionalized enzyme-cleavable peptide monomer comprises (MAA)-Gly-Pro-Gln-Gly-Ile-Ala-Gly-Gln-NH₂ (SEQ ID NO:36), (MAA)-Pro-Ser-Tyr-Phe-Leu-Asn-Ala-Gly-NH₂ (SEQ ID NO:37), (MAA)-Gly-Pro-Leu-Gly-Met-Arg-Gly-Leu-NH₂ (SEQ ID NO:38), (MAA)-Gly-Gly-Pro-Leu-Gly-Pro-Pro-Gly-NH₂ (SEQ ID NO:39).

32. The apparatus of any one of claims 1-6 wherein the functionalized enzyme-cleavable peptide monomer comprises a cathepsin G-cleavable sequence.

33. The apparatus of claim 32 wherein the functionalized enzyme-cleavable peptide monomer comprises (MAA)-Leu-Leu-Ser-Ala-Leu-Gln-NH₂ (SEQ ID NO:40) or (MAA)-Thr-Leu-Leu-Ser-Ala-Leu-Gln-NH₂ (SEQ ID NO:41).

34. The apparatus of any one of claims 1-6 wherein the functionalized enzyme-cleavable peptide monomer comprises a cathepsin D-cleavable sequence.

35. The apparatus of claim 34 wherein the functionalized enzyme-cleavable peptide monomer comprises (MAA)-Glu-Glu-Lys-Pro-Ile-Met-Phe-Phe-Arg-Leu-Leu-Gly-Lys-Lys-NH₂(SEQ ID NO:42), (MAA)-Glu-Asp-Lys-Pro-Ile-Leu-Phe-Phe-Arg-Leu-Gly-Lys-NH₂ (SEQ ID NO:43) or (MAA)-Glu-Glu-Lys-Pro-Ile-Ser-Phe-Phe-Arg-Leu-Gly-Lys-NH₂ (SEQ ID NO:44).

36. The apparatus of any one of claims 1-6 wherein the functionalized enzyme-cleavable peptide monomer comprises Cathepsin B cleavable sequence.

37. The apparatus of claim 36 wherein the functionalized enzyme-cleavable peptide monomer comprises (MAA)-Arg-Leu-Arg-Gly-Phe-Glu-NH₂ (SEQ ID NO:45), or (MAA)-Arg-Ile-Ile-Glu-Gly-Ile-Glu-NH₂ (SEQ ID NO:46).

38. The apparatus of any one of claims 1-37 wherein the cell adhesive peptide is selected from the group consisting of KGGGQKCIVQTTSWSQCSKS (SEQ ID NO:47); GGGQKCIVQTTSWSQCSKS(SEQ ID NO:48); KYGLALERKDHSG (SEQ ID NO:49); YGLALERKDHSG (SEQ ID NO:50); KGGSINNNRHSIYITRFGNMGS (SEQ ID NO:51); GGSINNNRHSIYITRFGNMGS (SEQ ID NO:52); KGGTWYKIAFQRNRK (SEQ ID NO:53); GGTWYKIAFQRNRK (SEQ ID NO:54); KGGTSIKIRGTYSER (SEQ ID NO:55); GGTSIKIRGTYSER (SEQ ID NO:56); KYGTDIRVTLNRLNTF (SEQ ID NO:57); YGTDIRVTLNRLNTF (SEQ ID NO:58); KYGSETTVKYIFRLHE (SEQ ID NO:59); YGSETTVKYIFRLHE(SEQ ID NO:60); KYGKAFDITYVRLKF (SEQ ID NO:61); YGKAFDITYVRLKF(SEQ ID NO:62); KYGAASIKVAVSADR (SEQ ID NO:63); YGAASIKVAVSADR(SEQ ID NO:64); KGGNGEPRGDTYRAY(SEQ ID NO:65); GGNGEPRGDTYRAY (SEQ ID NO:66) CGGNGEPRGDTRAY (SEQ ID NO:67); GGNGEPRGDTRAY (SEQ ID NO:68); KYGRKRLQVQLSIRT (SEQ ID NO:69); YGRKRLQVQLSIRT(SEQ ID NO:70); KGGRNIAEIIKDI (SEQ ID NO:71); GGRNIAEIIKDI (SEQ ID NO:72); KGGPQVTRGDVFTMP (SEQ ID NO:73); GGPQVTRGDVFTMP(SEQ ID NO:74); GGPQVTRGDVFTMPK (SEQ ID NO:75); GRGDSPK (SEQ ID NO:76); KGGAVTGRGDSPASS(SEQ ID NO:77); GGAVTGRGDSPASS (SEQ ID NO:78); XaaPQVTRGNVFTMP (SEQ ID NO:79); RGDYK (SEQ ID NO:80), GGVTRGNVFTMP(SEQ ID NO:81), where the peptide sequences may be linear or cyclic, or combinations.


SEQUENCE LISTING (81 sequences)

SEQ ID NO: 1 - protein, 2 aa, Artificial Sequence
  Description of Artificial Sequence Synthetic peptide
KK

SEQ ID NO: 2 - protein, 2 aa, Artificial Sequence
  Description of Artificial Sequence Synthetic peptide
KK

SEQ ID NO: 3 - protein, 3 aa, Artificial Sequence
  Description of Artificial Sequence Synthetic peptide
RRK

SEQ ID NO: 4 - protein, 3 aa, Artificial Sequence
  Description of Artificial Sequence Synthetic peptide
KKK

SEQ ID NO: 5 - protein, 2 aa, Artificial Sequence
  Description of Artificial Sequence Synthetic peptide
RR

SEQ ID NO: 6 - protein, 6 aa, Artificial Sequence
  Description of Artificial Sequence Synthetic peptide
FARARD

SEQ ID NO: 7 - protein, 6 aa, Artificial Sequence
  Description of Artificial Sequence Synthetic peptide
FARIRD

SEQ ID NO: 8 - protein, 4 aa, Artificial Sequence
  Description of Artificial Sequence Synthetic peptide
AYAF

SEQ ID NO: 9 - protein, 6 aa, Artificial Sequence
  Description of Artificial Sequence Synthetic peptide
ALFALR

SEQ ID NO: 10 - protein, 7 aa, Artificial Sequence
  Description of Artificial Sequence Synthetic peptide
AAAALFR

SEQ ID NO: 11 - protein, 4 aa, Artificial Sequence
  Description of Artificial Sequence Synthetic peptide
AAPA

SEQ ID NO: 12 - protein, 4 aa, Artificial Sequence
  Description of Artificial Sequence Synthetic peptide
AAPV

SEQ ID NO: 13 - protein, 4 aa, Artificial Sequence
  Description of Artificial Sequence Synthetic peptide
AAPM

SEQ ID NO: 14 - protein, 6 aa, Artificial Sequence
  Description of Artificial Sequence Synthetic peptide
REHVIF

SEQ ID NO: 15 - protein, 6 aa, Artificial Sequence
  Description of Artificial Sequence Synthetic peptide
AFELFR

SEQ ID NO: 16 - protein, 6 aa, Artificial Sequence
  Description of Artificial Sequence Synthetic peptide
AHFFRL

SEQ ID NO: 17 - protein, 4 aa, Artificial Sequence
  Description of Artificial Sequence Synthetic peptide
KTYK

SEQ ID NO: 18 - protein, 4 aa, Artificial Sequence
  Description of Artificial Sequence Synthetic peptide
KTFK

SEQ ID NO: 19 - protein, 4 aa, Artificial Sequence
  Description of Artificial Sequence Synthetic peptide
KTWK

SEQ ID NO: 20 - protein, 4 aa, Artificial Sequence
  Description of Artificial Sequence Synthetic peptide
KTSK

SEQ ID NO: 21 - protein, 4 aa, Artificial Sequence
  Description of Artificial Sequence Synthetic peptide
FTYK

SEQ ID NO: 22 - protein, 4 aa, Artificial Sequence
  Description of Artificial Sequence Synthetic peptide
LTFK

SEQ ID NO: 23 - protein, 4 aa, Artificial Sequence
  Description of Artificial Sequence Synthetic peptide
LEFK

SEQ ID NO: 24 - protein, 4 aa, Artificial Sequence
  Description of Artificial Sequence Synthetic peptide
LTPR

SEQ ID NO: 25 - protein, 4 aa, Artificial Sequence
  Description of Artificial Sequence Synthetic peptide
LTPR

SEQ ID NO: 26 - protein, 4 aa, Artificial Sequence
  Description of Artificial Sequence Synthetic peptide
VTPR

SEQ ID NO: 27 - protein, 4 aa, Artificial Sequence
  Description of Artificial Sequence Synthetic peptide
LTLR

SEQ ID NO: 28 - protein, 4 aa, Artificial Sequence
  Description of Artificial Sequence Synthetic peptide
LGVR

SEQ ID NO: 29 - protein, 4 aa, Artificial Sequence
  Description of Artificial Sequence Synthetic peptide
GGVR

SEQ ID NO: 30 - protein, 10 aa, Artificial Sequence
  Description of Artificial Sequence Synthetic peptide
VDVADVDVAD

SEQ ID NO: 31 - protein, 8 aa, Artificial Sequence
  Description of Artificial Sequence Synthetic peptide
DEVDDEVD

SEQ ID NO: 32 - protein, 7 aa, Artificial Sequence
  Description of Artificial Sequence Synthetic peptide
AAADAAD

SEQ ID NO: 33 - protein, 7 aa, Artificial Sequence
  Description of Artificial Sequence Synthetic peptide
LEHDAAD

SEQ ID NO: 34 - protein, 7 aa, Artificial Sequence
  Description of Artificial Sequence Synthetic peptide
VEIDAAD

SEQ ID NO: 35 - protein, 3 aa, Artificial Sequence
  Description of Artificial Sequence Synthetic peptide
DDD

SEQ ID NO: 36 - protein, 8 aa, Artificial Sequence
  Description of Artificial Sequence Synthetic peptide
GPQGIAGQ

SEQ ID NO: 37 - protein, 8 aa, Artificial Sequence
  Description of Artificial Sequence Synthetic peptide
PSYFLNAG

SEQ ID NO: 38 - protein, 8 aa, Artificial Sequence
  Description of Artificial Sequence Synthetic peptide
GPLGMRGL

SEQ ID NO: 39 - protein, 8 aa, Artificial Sequence
  Description of Artificial Sequence Synthetic peptide
GGPLGPPG

SEQ ID NO: 40 - protein, 6 aa, Artificial Sequence
  Description of Artificial Sequence Synthetic peptide
LLSALQ

SEQ ID NO: 41 - protein, 7 aa, Artificial Sequence
  Description of Artificial Sequence Synthetic peptide
TLLSALQ

SEQ ID NO: 42 - protein, 14 aa, Artificial Sequence
  Description of Artificial Sequence Synthetic peptide
EEKPIMFFRLLGKK

SEQ ID NO: 43 - protein, 12 aa, Artificial Sequence
  Description of Artificial Sequence Synthetic peptide
EDKPILFFRLGK

SEQ ID NO: 44 - protein, 12 aa, Artificial Sequence
  Description of Artificial Sequence Synthetic peptide
EEKPISFFRLGK

SEQ ID NO: 45 - protein, 6 aa, Artificial Sequence
  Description of Artificial Sequence Synthetic peptide
RLRGFE

SEQ ID NO: 46 - protein, 7 aa, Artificial Sequence
  Description of Artificial Sequence Synthetic peptide
RIIEGIE

SEQ ID NO: 47 - protein, 20 aa, Artificial Sequence
  Description of Artificial Sequence Synthetic peptide
KGGGQKCIVQTTSWSQCSKS

SEQ ID NO: 48 - protein, 19 aa, Artificial Sequence
  Description of Artificial Sequence Synthetic peptide
GGGQKCIVQTTSWSQCSKS

SEQ ID NO: 49 - protein, 13 aa, Artificial Sequence
  Description of Artificial Sequence Synthetic peptide
KYGLALERKDHSG

SEQ ID NO: 50 - protein, 12 aa, Artificial Sequence
  Description of Artificial Sequence Synthetic peptide
YGLALERKDHSG

SEQ ID NO: 51 - protein, 23 aa, Artificial Sequence
  Description of Artificial Sequence Synthetic peptide
KGGSINNNRWHSIYITRFGNMGS

SEQ ID NO: 52 - protein, 22 aa, Artificial Sequence
  Description of Artificial Sequence Synthetic peptide
GGSINNNRWHSIYITRFGNMGS

SEQ ID NO: 53 - protein, 15 aa, Artificial Sequence
  Description of Artificial Sequence Synthetic peptide
KGGTWYKIAFQRNRK

SEQ ID NO: 54 - protein, 14 aa, Artificial Sequence
  Description of Artificial Sequence Synthetic peptide
GGTWYKIAFQRNRK

SEQ ID NO: 55 - protein, 15 aa, Artificial Sequence
  Description of Artificial Sequence Synthetic peptide
KGGTSIKIRGTYSER

SEQ ID NO: 56 - protein, 14 aa, Artificial Sequence
  Description of Artificial Sequence Synthetic peptide
GGTSIKIRGTYSER

SEQ ID NO: 57 - protein, 16 aa, Artificial Sequence
  Description of Artificial Sequence Synthetic peptide
KYGTDIRVTLNRLNTF

SEQ ID NO: 58 - protein, 15 aa, Artificial Sequence
  Description of Artificial Sequence Synthetic peptide
YGTDIRVTLNRLNTF

SEQ ID NO: 59 - protein, 16 aa, Artificial Sequence
  Description of Artificial Sequence Synthetic peptide
KYGSETTVKYIFRLHE

SEQ ID NO: 60 - protein, 15 aa, Artificial Sequence
  Description of Artificial Sequence Synthetic peptide
YGSETTVKYIFRLHE

SEQ ID NO: 61 - protein, 15 aa, Artificial Sequence
  Description of Artificial Sequence Synthetic peptide
KYGKAFDITYVRLKF

SEQ ID NO: 62 - protein, 14 aa, Artificial Sequence
  Description of Artificial Sequence Synthetic peptide
YGKAFDITYVRLKF

SEQ ID NO: 63 - protein, 15 aa, Artificial Sequence
  Description of Artificial Sequence Synthetic peptide
KYGAASIKVAVSADR

SEQ ID NO: 64 - protein, 14 aa, Artificial Sequence
  Description of Artificial Sequence Synthetic peptide
YGAASIKVAVSADR

SEQ ID NO: 65 - protein, 15 aa, Artificial Sequence
  Description of Artificial Sequence Synthetic peptide
KGGNGEPRGDTYRAY

SEQ ID NO: 66 - protein, 14 aa, Artificial Sequence
  Description of Artificial Sequence Synthetic peptide
GGNGEPRGDTYRAY

SEQ ID NO: 67 - protein, 14 aa, Artificial Sequence
  Description of Artificial Sequence Synthetic peptide
CGGNGEPRGDTRAY

SEQ ID NO: 68 - protein, 13 aa, Artificial Sequence
  Description of Artificial Sequence Synthetic peptide
GGNGEPRGDTRAY

SEQ ID NO: 69 - protein, 15 aa, Artificial Sequence
  Description of Artificial Sequence Synthetic peptide
KYGRKRLQVQLSIRT

SEQ ID NO: 70 - protein, 14 aa, Artificial Sequence
  Description of Artificial Sequence Synthetic peptide
YGRKRLQVQLSIRT

SEQ ID NO: 71 - protein, 13 aa, Artificial Sequence
  Description of Artificial Sequence Synthetic peptide
KGGRNIAEIIKDI

SEQ ID NO: 72 - protein, 12 aa, Artificial Sequence
  Description of Artificial Sequence Synthetic peptide
GGRNIAEIIKDI

SEQ ID NO: 73 - protein, 15 aa, Artificial Sequence
  Description of Artificial Sequence Synthetic peptide
KGGPQVTRGDVFTMP

SEQ ID NO: 74 - protein, 14 aa, Artificial Sequence
  Description of Artificial Sequence Synthetic peptide
GGPQVTRGDVFTMP

SEQ ID NO: 75 - protein, 15 aa, Artificial Sequence
  Description of Artificial Sequence Synthetic peptide
GGPQVTRGDVFTMPK

SEQ ID NO: 76 - protein, 7 aa, Artificial Sequence
  Description of Artificial Sequence Synthetic peptide
GRGDSPK

SEQ ID NO: 77 - protein, 15 aa, Artificial Sequence
  Description of Artificial Sequence Synthetic peptide
KGGAVTGRGDSPASS

SEQ ID NO: 78 - protein, 14 aa, Artificial Sequence
  Description of Artificial Sequence Synthetic peptide
GGAVTGRGDSPASS

SEQ ID NO: 79 - protein, 13 aa, Artificial Sequence
  Description of Artificial Sequence Synthetic peptide
XPQVTRGNVFTMP

SEQ ID NO: 80 - protein, 5 aa, Artificial Sequence
  Description of Artificial Sequence Synthetic peptide
RGDYK

SEQ ID NO: 81 - protein, 12 aa, Artificial Sequence
  Description of Artificial Sequence Synthetic peptide
GGVTRGNVFTMP